(19) United States
(12) Patent Application Publication
(10) Pub. No.: US 2025/0031954 A1
(43) Pub. Date: Jan. 30, 2025

(54) CONTROL APPARATUS FOR MEDICAL IMAGE SHOOTING APPARATUS AND MEDICAL IMAGE SHOOTING SYSTEM

(71) Applicant: CANON KABUSHIKI KAISHA, Tokyo (JP)

(72) Inventors: KEISUKE OYAIZU, Tokyo (JP); MAKIKO UENO, Kanagawa (JP); SHINYA TANAKA, Tokyo (JP)

(21) Appl. No.: 18/912,314

(22) Filed: Oct. 10, 2024

Related U.S. Application Data
(63) Continuation of application No. PCT/JP2023/014828, filed on Apr. 12, 2023.

(30) Foreign Application Priority Data
Apr. 13, 2022 (JP) 2022-066336
Mar. 28, 2023 (JP) 2023-052456

(51) Int. Cl. A61B 3/00; A61B 3/10; A61B 3/14

(52) U.S. Cl. CPC A61B 3/0033; A61B 3/005; A61B 3/102; A61B 3/14

(57) ABSTRACT

A touch panel terminal which communicates with a medical image shooting apparatus, in which the touch panel terminal includes a touch panel unit to perform display for a touch operation and accept a touch operation, a control unit to generate a drive instruction signal for instructing drive of the medical image shooting apparatus based on the touch operation accepted by the touch panel unit, and a signal transmission unit configured to transmit the drive instruction signal to the medical image shooting apparatus, the touch panel unit accepts a first touch operation and a second touch operation, the first touch operation is a touch operation of enabling an acceptance of the second touch operation, and the signal transmission unit transmits, based on the second touch operation, a drive instruction signal for changing a relative position of the subject and the medical image shooting apparatus to the medical image shooting apparatus.

### CROSS-REFERENCE TO RELATED APPLICATIONS

This application is a Continuation of International Patent Application No. PCT/JP2023/014828, filed Apr. 12, 2023, which claims the benefit of Japanese Patent Application No. 2022-066336, filed Apr. 13, 2022, and Japanese Patent Application No. 2023-052456, filed Mar. 28, 2023, all of which are hereby incorporated by reference herein in their entirety.

### TECHNICAL FIELD

The present invention relates to a control apparatus for a medical image shooting apparatus and also relates to a medical image shooting system.

### BACKGROUND ART

PTL 1 discloses a technique of avoiding an erroneous operation by performing operation control in a case where a threshold number or more of touched points are detected for a threshold time period or longer in an operation on a touch panel monitor of an ultrasonic diagnosis apparatus.

### CITATION LIST

### Patent Literature

PTL 1 Japanese Patent Laid-Open No. 2017-74213

However, according to PTL 1, in a case where the threshold number of touched points is small or the threshold time period is short, the operation control is frequently performed, and operability is lowered. On the other hand, in a case where the threshold number of touched points is set to be large or the threshold time period is set to be long, control of avoiding an erroneous touch operation may not be appropriately performed, and a medical image shooting apparatus subjected to the operation may erroneously drive, so that it is difficult to appropriately configure settings to avoid the erroneous touch operation such as settings on the threshold number of touched points and the threshold time period.

### SUMMARY OF INVENTION

A control apparatus for a medical image shooting apparatus according to a first embodiment of the present invention is a control apparatus for a medical image shooting apparatus, including:
a control terminal configured to control a medical image shooting apparatus that shoots a subject; and
a display apparatus which is capable of communicating with the control terminal and which is portable, in which
the display apparatus includes
a touch panel unit configured to perform display for a touch operation and accept a touch operation,
a signal generation unit configured to generate a drive instruction signal for instructing drive of a drive unit included in the medical image shooting apparatus based on the touch operation accepted by the touch panel unit, and
a signal transmission unit configured to transmit the drive instruction signal to the control terminal that controls the medical image shooting apparatus,
the control terminal includes
a signal reception unit configured to receive the drive instruction signal, and
a control unit configured to perform control to cause the drive unit of the medical image shooting apparatus to drive based on the drive instruction signal received by the signal reception unit,
the touch panel unit accepts a first touch operation and a second touch operation,
the first touch operation is a touch operation of enabling an acceptance of the second touch operation, and
the signal transmission unit transmits, based on the second touch operation accepted by the touch panel unit, a drive instruction signal for changing a relative position of the subject and the medical image shooting apparatus to the control terminal.

Further features of the present invention will become apparent from the following description of exemplary embodiments with reference to the attached drawings.

### BRIEF DESCRIPTION OF DRAWINGS

FIG. 1 illustrates a schematic configuration of an ophthalmic shooting system and an example of an optical layout of a first embodiment.

FIG. 2A illustrates a schematic configuration example of a control terminal of the first embodiment.

FIG. 2B illustrates a schematic configuration example of a touch panel terminal in a transformation example.

FIG. 3 illustrates an example of a shooting flow of the first embodiment.

FIG. 4A illustrates an example of a screen for performing a chin rest adjustment according to the first embodiment.

FIG. 4B illustrates an example of the screen for performing the chin rest adjustment according to the first embodiment.

FIG. 4C illustrates an example of a screen for performing a head position adjustment.

FIG. 4D illustrates an example of a screen for instructing shooting start.

FIG. 5A illustrates an example of a screen during tomographic image optimization of the first embodiment.

FIG. 5B illustrates an example of the screen during the tomographic image optimization of the first embodiment.

FIG. 5C illustrates an example of the screen during the tomographic image optimization of the first embodiment.

FIG. 6A illustrates an example of a screen before eye fundus image optimization of the first embodiment is performed.

FIG. 6B illustrates an example of a screen during the eye fundus image optimization of the first embodiment.

FIG. 6C illustrates an example of the screen during the eye fundus image optimization of the first embodiment.

FIG. 6D illustrates an example of the screen during the eye fundus image optimization of the first embodiment.

FIG. 7 illustrates an example of a setting table for an operation mode of a second embodiment.

FIG. 8 illustrates a schematic configuration example of an X-ray image shooting system of a fourth embodiment.

FIG. 9 illustrates an example of the shooting flow of the fourth embodiment.

FIG. 10A illustrates an example of a screen for performing alignment between a subject and an X-ray image shooting apparatus according to the fourth embodiment.

FIG. 10B illustrates an example of the screen for performing the alignment between the subject and the X-ray image shooting apparatus according to the fourth embodiment.

FIG. 11 illustrates a schematic configuration example of an X-ray image shooting system of a fifth embodiment.

FIG. 12 illustrates the shooting flow of the fifth embodiment.

FIG. 13A illustrates an example of a new examination input screen of the fifth embodiment.

FIG. 13B illustrates an example of the new examination input screen of the fifth embodiment.

FIG. 13C illustrates an example of the new examination input screen of the fifth embodiment.

FIG. 14A illustrates an example of a shooting screen.

FIG. 14B illustrates an example of a password input screen.

### DESCRIPTION OF EMBODIMENTS

Hereinafter, illustrative embodiments for carrying out the present invention will be described in detail with reference to the drawings. It is however noted that dimensions, shapes, positions of components, and the like which will be described in the following embodiments are optional and can be changed according to a configuration of an apparatus to which the present invention is applied or various conditions. In addition, in the drawings, the same reference signs are used across the drawings to indicate identical or functionally similar elements.

### First Embodiment

Hereinafter, a first embodiment will be described with reference to the drawings.

### Apparatus Configuration

A medical image shooting apparatus to be used in the present embodiment is an ophthalmic shooting apparatus and is capable of capturing an eye fundus image and a tomographic image.

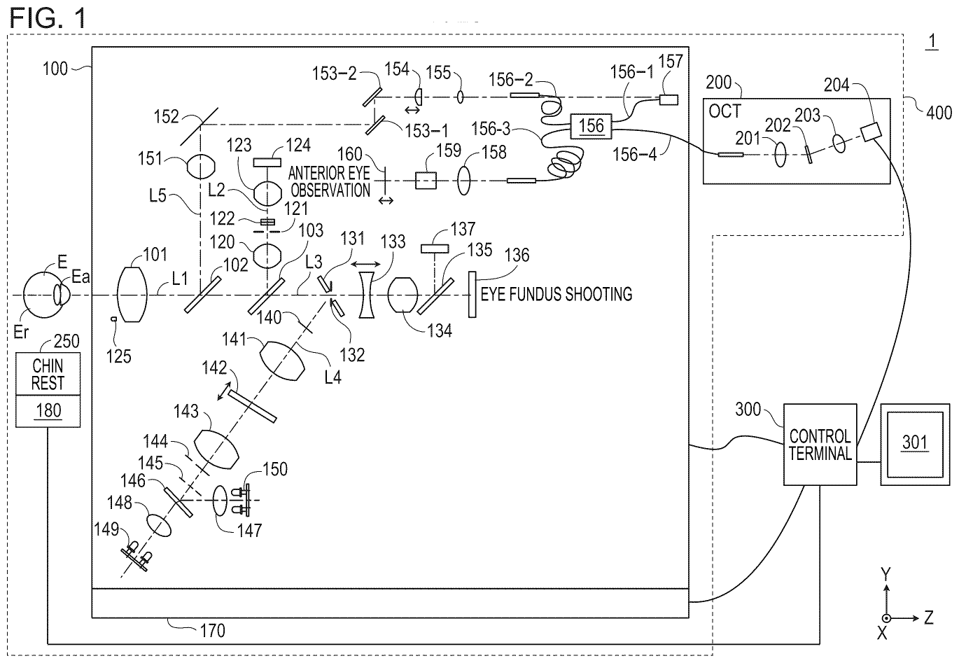

FIG. 1 is a schematic diagram of an apparatus configuration of an ophthalmic shooting system 1 according to the present embodiment.

The ophthalmic shooting system 1 of the present embodiment is provided with an ophthalmic shooting apparatus 400 including an optical head unit 100, a spectroscope 200, and a chin rest 250, and a control apparatus for the ophthalmic shooting apparatus 400 which includes a display apparatus 301 and a control terminal 300. It is noted that the optical head unit 100 and the spectroscope 200 can be collectively referred to as a shooting unit, and a head drive unit 170 configured to drive the optical head unit 100 can be referred to as a shooting unit drive unit.

### Configuration of the Display Apparatus 301

First, the display apparatus 301 will be described.

The display apparatus 301 is a user interface which is portable and capable of communicating with the ophthalmic shooting apparatus 400 and is a display apparatus including a touch panel unit configured to generate a drive instruction signal for driving the ophthalmic shooting apparatus 400.

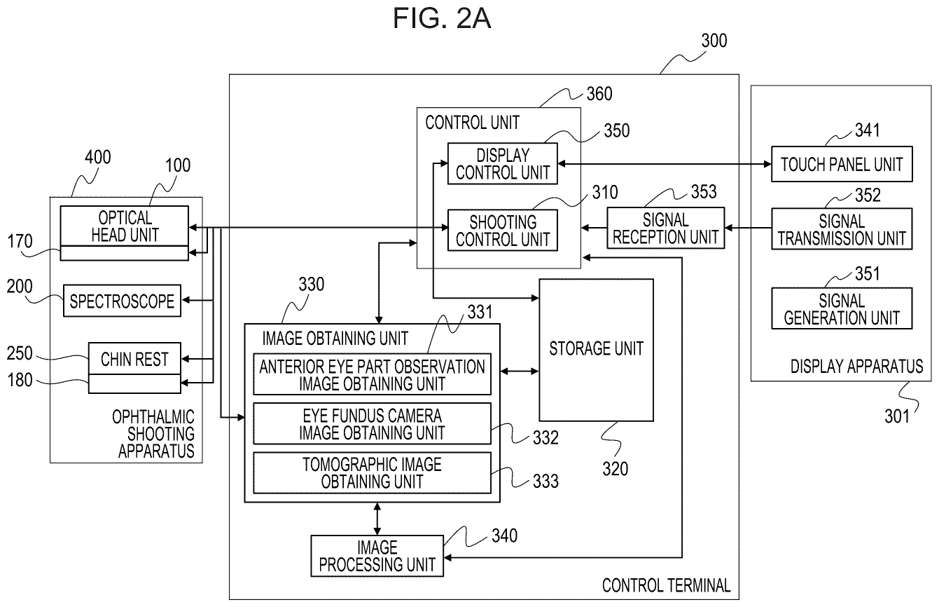

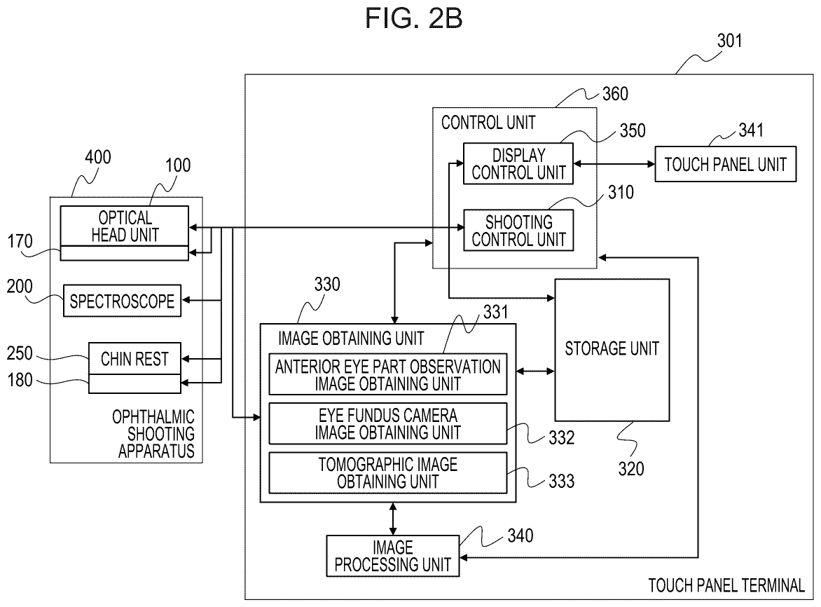

As illustrated in FIG. 2A, the display apparatus 301 includes a touch panel unit 341, a signal generation unit 351, and a signal transmission unit 352. A display control unit 350 displays an anterior eye part observation image, an eye fundus infrared observation image, an eye fundus image, and a tomographic image (B-scan image or the like) obtained by the ophthalmic shooting apparatus 400. The touch panel unit 341 has a function as an interface for operations and a function as a display unit configured to display a shot image and the like on a screen on the display apparatus 301. In the touch panel unit 341, when a drive instruction for driving the ophthalmic shooting apparatus 400 is accepted, the signal generation unit 351 functions as a drive instruction signal generation unit configured to drive the ophthalmic shooting apparatus 400 and generates a drive instruction signal. Next, the signal transmission unit 352 transmits the generated drive instruction signal to the control terminal 300 of the ophthalmic shooting apparatus 400. After the drive instruction signal transmitted by the signal transmission unit 352 included in the display apparatus 301 is received by a signal reception unit 353 included in the control terminal 300, a shooting control unit 310 drives the ophthalmic shooting apparatus 400 following the drive instruction signal.

### Configuration of the Ophthalmic Shooting Apparatus 400

Next, a configuration of the optical head unit 100, the spectroscope 200, and the chin rest 250 which constitute the ophthalmic shooting apparatus 400 illustrated in FIG. 1 will be described in sequence.

The optical head unit 100 includes a measurement optical system configured to capture a front image and a tomographic image of an anterior eye part Ea of a subject's eye E and an eye fundus Ef of the subject's eye. Hereinafter, various optical systems of the optical head unit 100 will be described.

An objective lens 101 is installed so as to face the subject's eye E. A first dichroic mirror 102 and a second dichroic mirror 103 which function as optical path separation units are arranged on an optical axis L1 of the objective lens 101. By these dichroic mirrors, an optical path (optical axis L2) of an anterior eye part observation system, an optical path (optical axis L3) of an eye fundus shooting system, and an optical path (optical axis L5) of an optical coherence tomography (OCT) are branched for each wavelength band.

A lens 120, an aperture 121, a prism 122, a lens 123, and an image sensor 124 are arranged on the optical axis L2 in a reflection direction of the second dichroic mirror 103. The image sensor 124 is a monochrome sensor with sensitivity to an infrared region. An anterior eye part observation optical system configured to observe an anterior eye part is constituted by these optical members and the like arranged on the optical axis L2. The image sensor 124 is connected to the control terminal 300. Each of pixel values obtained by the image sensor 124 is output to the display apparatus 301 via the control terminal 300. In addition, a light source 125 for anterior eye part observation which is arranged near the objective lens 101 illuminates the anterior eye part of the subject's eye E.

A holed mirror 131, a shooting aperture 132, a focus lens 133 and an imaging lens 134, a third dichroic mirror 135, and an image sensor 136 are arranged on the optical axis L3 in a transmission direction of the second dichroic mirror 103. An optical system for observing and shooting an eye fundus part is constituted. The holed mirror 131 has an opening in a central part. The focus lens 133 adjusts a focus by moving its position on the optical axis L3. The optical path on the optical axis L3 is branched for each wavelength band by the third dichroic mirror 135 into an optical path leading to the image sensor 136 and an optical path leading to a fixation lamp 137. The image sensor 136 is a sensor which has sensitivity to visible light and infrared light and serves for both video observation and still image shooting. The fixation lamp 137 projects visible light to help subject's fixation.

A corneal baffle 140, a relay lens 141, a focus indicator unit 142, a lens 143, and a ring slit 144 are arranged on an optical axis L4 in a reflection direction of the holed mirror 131 in the stated order. The corneal baffle 140 has a shading point at a center. The ring slit 144 has a ring-shaped slit opening. In addition, a crystal baffle 145 serving as a light shielding member having a shading point and a dichroic mirror 146 having properties to transmit infrared light and reflect visible light are arranged on the optical axis L4. The focus indicator unit 142 is movable along the optical axis L4 and can be removably inserted to the optical axis L4 from above.

A condenser lens 147 and the white light emitting diode (LED) light source 150 are arranged in a reflection direction of the dichroic mirror 146. A white LED light source 150 is a light source for shooting in which a plurality of white LEDs configured to emit visible pulsed light are arranged. The condenser lens 148 and an infrared LED light source 149 which serves as illumination means for illuminating a predetermined site with near infrared or infrared light are arranged in a transmission direction of the dichroic mirror 146. The infrared LED light source 149 is an observation light source in which a plurality of infrared LEDs configured to emit constant infrared light are arranged. The objective lens 101 and the dichroic mirror 146, optical members between these components, and the condenser lens 147 and the condenser lens 148 constitute an illumination optical system configured to illuminate an eye fundus. The eye fundus of the subject's eye is illuminated with light of the white LED light source 150 or the infrared LED light source 149 via this illumination optical system.

A lens 151, a mirror 152, an OCTX scanner 153-1, an OCTY scanner 153-2, and lenses 154 and 155 are arranged on the optical axis L5 in a reflection direction of the first dichroic mirror 102. The OCTX scanner 153-1 and the OCTY scanner 153-2 are constituted by galvanometer mirrors, for example, and function as scanning means for two-dimensionally scanning the subject's eye with measuring light. It is noted that the OCTX scanner 153-1 and the OCTY scanner 153-2 respectively perform scanning with the measuring light in a main scanning direction and a sub scanning direction orthogonal to the main scanning direction, but the scanning direction is not limited to this.

A measurement light source 157 serves as a light source configured to emit light for obtaining measuring light to be incident on a measuring light path. In the case of the present embodiment, the measuring light in the OCT optical system is emitted while a fiber end is set as a light source, and the fiber end has an optical conjugate relationship with an eye fundus Er of the subject's eye E. The lens 154 is a lens for focus adjustment and is driven in an optical axis direction indicated by an arrow in the drawing by a motor which is not illustrated in the drawing. The focus adjustment for the measuring light is performed such that the measuring light emitted from the fiber end acting as the light source is imaged on the eye fundus Er. The lens 154 functioning as a focus adjustment unit is arranged between the fiber end serving as the measuring light source and the OCTX scanner 153-1 and the OCTY scanner 153-2 which function as a scanning unit. By the above-described focus adjustment, the image of the measuring light emitted from the fiber end can be imaged on the eye fundus Er of the subject's eye E, and it is possible for return light from the eye fundus Er to efficiently return to an optical fiber 156-2.

It is noted that in FIG. 1, an optical path between the OCTX scanner 153-1 and the OCTY scanner 153-2 is configured in a paper plane but is actually configured in a direction perpendicular to the paper plane.

Next, a configuration of an optical path from the measurement light source 157, a reference optical system, and the spectroscope 200 will be described.

The measurement light source 157, an optical coupler 156, optical fibers 156-1 to 156-4, a lens 158, a glass 159 for dispersion compensation, a reference mirror 160, and the spectroscope 200 constitute a Michelson interferometer system. The optical fibers 156-1 to 156-4 are optical fibers in a single mode which are connected to the optical coupler 156 to be integrated. Light emitted from a measurement light source 204 is guided to the optical coupler 156 via the optical fiber 156-1, and the light guided to the optical coupler 156 is split by the optical coupler 156 into measuring light on the optical fiber 156-2 side and reference light on the optical fiber 156-3 side. The measuring light is irradiated to the eye fundus Ef of the subject's eye E set as an observation target through the optical path of the above-described OCT optical system and reaches the optical coupler 156 again through the same optical path due to reflection and scattering by a retina.

On the other hand, the reference light reaches the reference mirror 160 via the optical fiber 156-3, the lens 158, and the glass 159 for dispersion compensation which is inserted to match the dispersion of the measuring light and the reference light, and the reference light is reflected. The reference light reflected by the reference mirror 160 returns through the same optical path and reaches the optical coupler 156 again. The reference light that has reached the optical coupler 156 again and the measuring light (return light) are combined by the optical coupler 156. Herein, when an optical path length of the measuring light and an optical path length of the reference light become substantially identical, an interference due to each light occurs by this combination. The reference mirror 160 is held such that its position is adjustable in the optical axis direction indicated by the arrow in the drawing by a motor and a drive mechanism which are not illustrated in the drawings. The optical path length of the reference light can be matched with the optical path length of the measuring light which changes depending on the subject's eye E by using this motor or the like. The obtained interference light is guided to the spectroscope 200 via the optical fiber 156-4.

The spectroscope 200 includes a lens 201, a diffraction grating 202, a lens 203, and a line sensor 204 serving as light reception means for receiving the return light from the subject's eye. The interference light emitted from the optical fiber 156-4 has turned to substantially parallel light via the lens 201 to be then split by the diffraction grating 202 and imaged by the lens 203 on the line sensor 204. Each of elements in the line sensor 204 outputs a signal according to the received light. The control terminal 300 samples this signal at predetermined timing by an image obtainment and generation unit 304 which will be described below and applies predetermined signal processing to the signal to generate a tomographic image.

Next, a surrounding area of the measurement light source 157 will be described. According to the present embodiment, a super luminescent diode (SLD) serving as a representative low coherence light source is used as the measurement light source 157. A central wavelength of the light to be emitted from the measurement light source 157 is 880 nm, and a wavelength bandwidth is approximately 60 nm. Herein, the bandwidth is an important parameter since the bandwidth impacts a resolution in an optical axis of a tomographic image to be obtained. In addition, the SLD is selected as a type of the light source herein, but any type may be used as long as low coherence light may be emitted, and amplified spontaneous emission (ASE) or the like can also be used. When it is considered that the eye is to be measured, with regard to the central wavelength of the measuring light, near-infrared light is appropriate. In addition, since the central wavelength impacts a resolution in a lateral direction of the tomographic image to be obtained, the wavelength is preferably as short as possible. Due to both of the reasons, according to the present embodiment, light with the central wavelength at 880 nm is used.

In addition, as will be described below, a chin rest drive unit 180 drives up and down to drive the chin rest 250 following an instruction of an operator from the touch panel unit 341, so that the subject's eye E is arranged at an appropriate position.

It is noted that according to the present embodiment, a Michelson interferometer is used as an interferometer, but a Mach-Zehnder interferometer may be used.

Furthermore, the optical head unit 100 is connected to the head drive unit 170. The head drive unit 170 is constituted by three motors which are not illustrated in the drawing and is configured to be able to move the optical head unit 100 in three-dimensional (X, Y, and Z) directions relative to the subject's eye E. With this configuration, it is possible to perform the alignment of the optical head unit 100 relative to the subject's eye E.

Next, with reference to FIG. 2A, a schematic configuration of the control terminal 300 will be described. The control terminal 300 is provided with a control unit 360 including the shooting control unit 310 and the display control unit 350, a storage unit 320, an image obtaining unit 330, and an image processing unit 340.

The shooting control unit 310 is connected to the optical head unit 100, the head drive unit 170 which drives the optical head unit 100, the spectroscope 200, the chin rest 250, the chin rest drive unit 180 which drives the chin rest 250, the storage unit 320, the image obtaining unit 330, and the display apparatus 301. The shooting control unit 310 performs shooting by controlling components in association with shooting (for example, the optical head unit 100, the head drive unit 170 which drives the optical head unit 100, the spectroscope 200, the chin rest 250, the chin rest drive unit 180 which drives the chin rest 250, and the storage unit 320) based on the drive instruction signal for driving the ophthalmic shooting apparatus 400 which has been generated by the display apparatus 301.

According to the present embodiment, a drive instruction signal transmitted from the signal transmission unit 352 included in the display apparatus 301 is received by the signal reception unit 353 included in the control terminal 300, and following the drive instruction signal, the shooting control unit 310 performs drive control to change a height of the chin rest 250 relative to the chin rest drive unit 180 so that a relative position of the subject's eye E relative to the optical head unit 100 is changed in an up and down direction. In addition, in order that the examination sequence read from the storage unit 320 is executed, the shooting control unit 310 controls the head drive unit 170 to perform alignment of the optical head unit 100 to the subject's eye and controls the optical head unit 100 and the spectroscope 200 to perform shooting.

In anterior eye part observation image shooting, the shooting control unit 310 controls the light source 125 for anterior eye part observation to emit light, and the image sensor 124 receives light and reads a received light signal. The read signal is transmitted to an anterior eye part observation image obtaining unit 331 included in the image obtaining unit 330.

In eye fundus infrared observation image shooting by an eye fundus camera, the shooting control unit 310 causes the infrared LED light source 149 to emit light, and for eye fundus observation and tracking, the eye fundus infrared observation image shooting is performed. The shooting control unit 310 drives the focus indicator unit 142 to obtain the diopter scale information of the subject's eye E, and then performs control to drive the focus lens 133 so as to match the diopter scale information. The image sensor 136 then receives light and reads a received light signal. The read signal is transmitted to an eye fundus camera image obtaining unit 332 included in the image obtaining unit 330. The eye fundus camera image obtaining unit 332 generates an eye fundus infrared observation image by using the image processing unit 340.

In eye fundus image shooting by an eye fundus camera, a position of the focus lens 133 is changed to be aligned with a diopter scale position obtained by applying a correction of aberration due to a difference in wavelengths of light sources to the diopter scale information of the subject's eye E which has been sensed by the infrared LED light source 149. Then, the white LED light source 150 is caused to emit visible pulsed light to be read by the image sensor 136 to read a signal. The read signal is transmitted to the eye fundus camera image obtaining unit 332 included in the image obtaining unit 330.

In the tomographic image shooting, the focus lens 154 is caused to drive based on the diopter scale information detected by the focus indicator unit 142, and scanning control signals are transmitted to the OCTX scanner 153-1 and the OCTY scanner 153-2, so that scanning is performed on the eye fundus Er of the subject's eye in the X direction and the Y direction with the measuring light from the measurement light source 157. Then, a signal received by the line sensor 204 is read. The read signal is transmitted to a tomographic image obtaining unit 333 included in the image obtaining unit 330. The tomographic image obtaining unit 333 performs Fourier transformation on the signal obtained from the line sensor 204 by using the image processing unit 340 and obtains an image of the subject's eye in a depth direction (Z direction) by converting obtained data into brightness or density information. Such a scanning method is called an A-scan, and a tomographic image to be obtained is called an A-scan image. A time period spent for the image processing by the image processing unit 340 is 20 microseconds or shorter. Since the data of the line sensor 204 is transmitted every 20 microseconds, when next data is obtained by the line sensor 204 while the image processing unit 340 performs the image processing, the A-scan can be performed every 20 microseconds.

By scanning with the measuring light for performing this A-scan by the OCTX scanner 153-1 and the OCTY scanner 153-2 in a predetermined transverse direction on the eye fundus Er of the subject's eye, it is possible to obtain a plurality of A-scan images. Then, in the image processing unit 340, the tomographic image is constituted from the plurality of A-scan images and scanning information.

The storage unit 320 stores the anterior eye part observation image, the eye fundus infrared observation image, the eye fundus image, and the tomographic image which are obtained by the image obtaining unit 330. In addition, the storage unit 320 stores an examination sequence which defines a series of control procedures in which the examination is executed multiple times, the generated image of the subject's eye, an analysis result of the image, an image capture condition at the time when the image is obtained, so-called patient information or the like related to the subject's eye, and various programs and the like used when the anterior eye part observation image shooting, the eye fundus infrared observation image shooting and the eye fundus image shooting by the eye fundus camera, and the tomographic image shooting which have been described above are controlled.

It is noted that the control terminal 300 described above may be configured by a module executed by a CPU or an MPU or may be configured by a circuit or the like which realizes a particular function such as an application-specific integrated circuit (ASIC). In addition, the storage unit 320 can be configured by a storage medium such as an optional memory or an optical disk.

### Shooting Flow

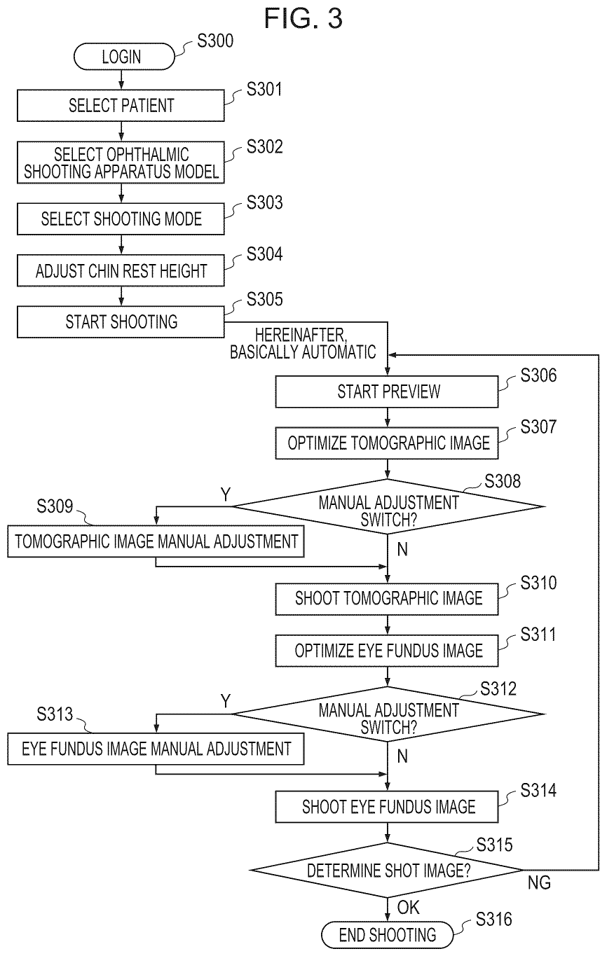

An example of a shooting flow in a case where the ophthalmic shooting apparatus that shoots the tomographic image and the eye fundus image is selected as a model of the ophthalmic shooting apparatus will be described with reference to FIG. 3.

First, the operator logs in to shooting software in S300.

In S301, a patient is selected on a patient list screen.

In S302, a model of the ophthalmic shooting apparatus is selected.

In S303, as a selection of a shooting mode, a selection of the subject's eye (eye to be shot) for selecting which eye to be shot out of the left eye and the right eye, a selection of a shooting site (a macula or an optic papilla of the eye fundus, or the like), and a selection of a scan pattern used when the tomographic shooting is carried out are performed. At this time, simultaneously, the fixation lamp for helping fixation of the subject in a direction appropriate to shooting of a desired shooting site is preferably turned on.

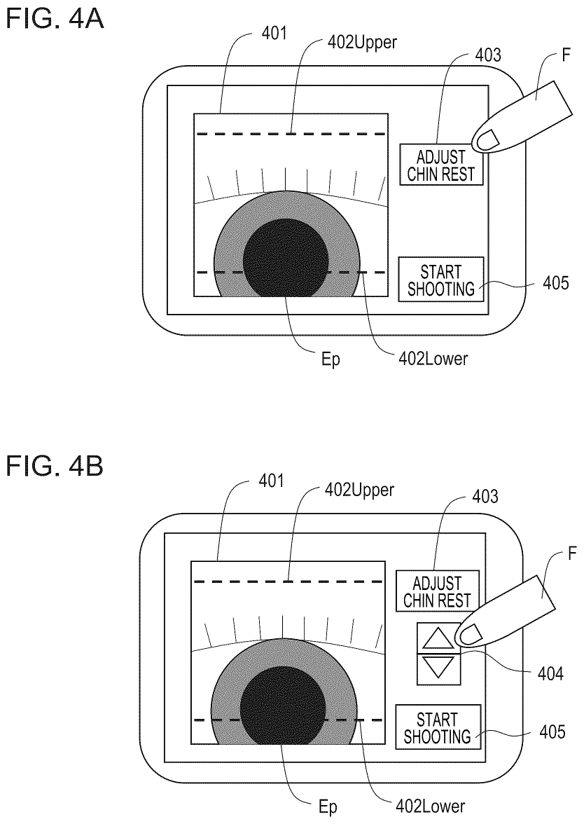

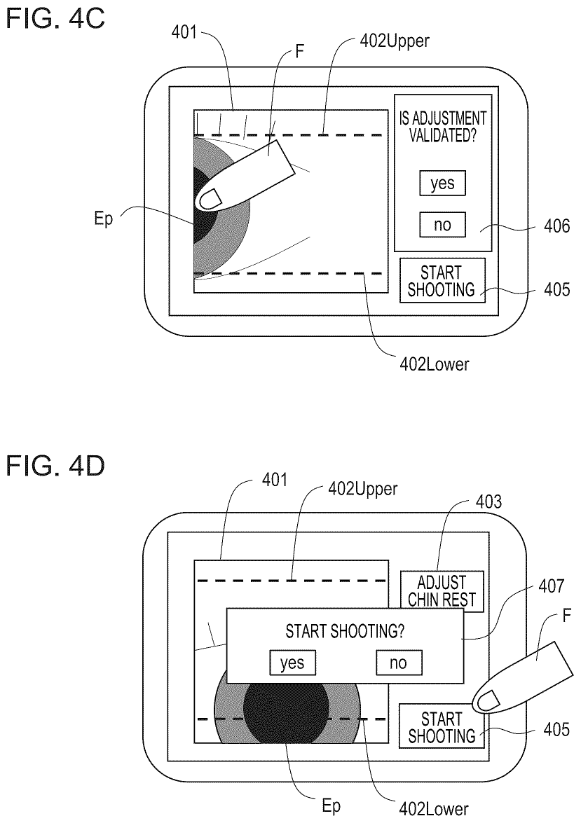

In S304, a chin rest height adjustment is performed. In the display apparatus 301, an anterior eye part observation image 401 is displayed as illustrated in FIGS. 4A to 4D on the touch panel unit 341 by the display control unit 350. In the example illustrated in FIGS. 4A to 4D, a pupil position arrangement area 402 is indicated on the anterior eye part observation image 401.

Then, in a case where a position of a pupil Ep is largely deviated vertically from the pupil position arrangement area 402, the operator first checks a position of the chin rest (250 in FIG. 1), and to adjust an up and down position of the chin rest when necessary, a touch operation (first touch operation) is performed on a chin rest adjustment button 403 that is a first touch operation region illustrated in FIG. 4A. With this configuration, as illustrated in FIG. 4B, up and down buttons 404 serving as a second touch operation region for performing a parameter setting of the chin rest adjustment are displayed. Then, when the operator performs a touch operation (second touch operation) on the up and down buttons 404, according to the number of touches on the up and down buttons 404 and the touch time period, the signal generation unit 351 generates a drive instruction signal for driving the chin rest drive unit 180 by a previously set amount. The generated drive instruction signal is transmitted by the signal transmission unit 352 from the display apparatus 301 to the control terminal 300, and based on the drive instruction signal, the shooting control unit 310 performs control to cause the chin rest drive unit 180 to drive, so that the chin rest drive unit 180 is driven. With this configuration, the operator can arrange the pupil Ep in the pupil position arrangement area 402 by performing the touch operation on both the chin rest adjustment button 403 and the up and down buttons 404. It is noted that a whole region of the touch operation region does not necessarily need to be a region for accepting an instruction through the touch operation. For example, such a configuration may be included in which a part of the region such as a screen for including a button for accepting the instruction through the touch operation is a region for accepting the instruction through the touch operation. In addition, the touch operation region can be called a touch panel region or a graphical user interface (GUI).

In this way, the position of the chin rest is adjusted through two-step operations including the first touch operation (touch operation on the chin rest adjustment button 403) for enabling the acceptance of the second touch operation and the second touch operation (touch operation on the up and down buttons 404) for performing the parameter setting for the chin rest adjustment which is displayed by the first touch operation.

At this time, when the touch operation is performed on the chin rest adjustment button 403 that is the first touch operation region, pulldown display of the up and down buttons 404 that are the second touch operation region for the chin rest adjustment may be performed near the chin rest adjustment button 403, and the adjustment may be performed through a touch operation (that is, a flick input) in which the touch operation (first touch operation) on the chin rest adjustment button 403 and the touch operation (second touch operation) on the up and down buttons 404 are continuous to each other.

In addition, in a case where the pupil position is largely deviated in a left and right direction as in FIG. 4C, a drive instruction can be issued by performing the touch operation on the touch panel unit 341 of the display apparatus 301 to drive the head drive unit 170 so that an adjustment in the left and right direction is performed. For example, similarly as in the chin rest adjustment button, a method of displaying a head adjustment button on the touch panel unit (341 in FIG. 1) on the display apparatus 301 to perform the touch operation (first touch operation) on the head adjustment button, so that pulldown display of left and right adjustment buttons of the head is performed to adjust a left and right position of the head by operating the left and right adjustment buttons (second touch operation), a method of performing the touch operation (first touch operation) to instruct drive of the head drive unit 170 and the drive amount on the anterior eye part observation image 401 and thereafter performing the touch operation (second touch operation) to permit the drive of the head drive unit 170 for the adjustment, and the like are conceivable.

In the case of the latter, for example, in the anterior eye part observation image 401 of FIG. 4C, control may be performed in a manner that the head drive unit 170 moves so that the touched site comes to the center of the image. More specifically, in the anterior eye part observation image 401 of FIG. 4C, when the first touch operation is performed to touch one point around the center of the pupil on the anterior eye part observation image 401 by a finger F, a screen (screen including a message for requesting a permission to perform the drive of the head drive unit 170 which has been set by the first touch operation) 406 for requesting a permission to perform the drive of the head drive unit 170 in the left and right direction is displayed on the touch panel unit 341 on the display apparatus 301. Herein, when the operator performs the touch operation of selecting “Yes” as the second touch operation, the drive instruction signal for instructing the drive of the head drive unit 170 is generated by the signal generation unit 351 and transmitted by the signal transmission unit 352 to the control terminal 300, so that the shooting control unit 310 performs control to cause the head drive unit 170 to drive based on the signal, and the adjustment in the left and right direction is executed.

It is noted that at this time, a configuration may be adopted in which when the screen 406 including the message for requesting the permission to perform the drive of the head drive unit 170 is displayed, the further first touch operation is not to be performed, and only the second touch operation is accepted. However, a configuration can be adopted in which even after the message 406 is displayed, the further first touch operation is accepted.

In such a case, for example, with regard to display of selection buttons YES/NO for performing the second touch operation, the display is performed at a position away from the anterior eye part observation image 401. That is, the first touch operation region and the second touch operation region are displayed in different positions of the touch panel unit 341, so that erroneous drive due to two consecutive unintended touches at nearby locations, that is, the erroneous touch operations can be further reduced.

In addition, herein, the example has been described in which after the first touch operation is accepted, the screen 406 which displays a message for requesting a permission to perform the drive set by the first touch operation is displayed, and after the second touch operation of selecting Yes is accepted, the head drive unit 170 is driven. However, a configuration is also conceivable in which a screen for requesting a permission to perform drive first is displayed, and after the first touch operation of selecting Yes on the screen is performed, when the second touch operation for driving the head drive unit 170 on the anterior eye part observation image 401 is performed, the drive instruction signal for causing the head drive unit 170 to drive is generated by the signal generation unit 351. In addition, by adopting a configuration in which when Yes is selected on the screen including the message for requesting the permission to perform the drive of the head drive unit 170, at the same time, pulldown display of the chin rest up and down buttons is performed, so that to carry out adjustments (up, down, left, and right adjustments) of the position of the subject's eye, it is possible to avoid requests of operations in multiple steps, and the adjustment can be performed without complicated operations.

As an example of still another method, instead of the adjustment method for the head position of driving the head drive unit 170 by the first touch operation and the second touch operation such that a position on which the touch operation has been performed (which can also be referred to as a site) comes to the center of the image, up, down, left and right arrow buttons may be displayed in a region connected to the touched position by the first touch operation on the pupil position arrangement area 402, and adjustments of the up, down, left and right positions of the optical head unit 100 may be performed by continuous flick operations (the first touch operation and the continuous second touch operation) on the arrow buttons. With such operations, it is also possible to realize smoother operations.

As described above, the control is performed by carrying out the drive to change the relative position of the subject's eye and the ophthalmic shooting apparatus such as the position adjustment of the chin rest or position adjustment of an optical head part through two-step operations (the first touch operation of enabling the acceptance of the second touch operation and the second touch operation). Thus, such a state hardly occurs where since the operator touches the touch panel unit of the display apparatus without an intention (erroneous touch operation) to cause the drive to change the relative position of the subject's eye and the ophthalmic shooting apparatus at unintended timing or unnecessarily. That is, through such two-step operations (the first touch operation of enabling the acceptance of the second touch operation and the second touch operation), it is possible to avoid a collision between the ophthalmic shooting apparatus 400 and the subject along with the change in the relative position of the subject's eye and the ophthalmic shooting apparatus which is caused by the erroneous touch operation, for example. It is noted that a concept of “changing the relative position of the subject's eye and the ophthalmic shooting apparatus” includes a concept of “changing a distance between the subject's eye and the shooting unit (herein, the optical head unit 100 and the spectroscope 200)”, and includes a concept of changing a distance between the subject's eye and a predetermined position on the shooting unit, for example.

In addition, with regard to a relationship between the first touch operation and the second touch operation, a configuration may be adopted in which the signal generation unit 351 generates the drive instruction signal by accepting the first touch operation, and the signal transmission unit 352 transmits the drive instruction signal by accepting the second touch operation. A configuration may also be adopted in which by the first touch operation and the second touch operation, the signal generation unit 351 generates a drive instruction signal, and the signal transmission unit 352 transmits the drive instruction signal.

Next, in S305, shooting is started. As illustrated in FIG. 4D, when the operator performs a touch operation of pressing a shooting start button 405 displayed on the touch panel unit 341 of the display apparatus 301, shooting start is executed. At this time too, when the operator performs the touch operation (first touch operation) of pressing the shooting start button 405, a screen 407 including a message for requesting a permission to perform shooting start is displayed on the touch panel unit 341 of the display apparatus 301. Then, in a case where the operator performs an operation of selecting Yes (second touch operation), a drive instruction signal for starting shooting preparation (adjustment for performing shooting) is generated by the signal generation unit 351 and transmitted by the signal transmission unit 352 from the display apparatus 301 to be received by the signal reception unit 353 included in the control terminal 300.

When the shooting start in S305 is executed, the shooting control unit 310 performs control to drive the head drive unit 170. Specifically, the optical head unit 100 is moved in the three-dimensional (X, Y, and Z) directions relative to the subject's eye E so as to arrange the optical head unit 100 to an appropriate position when the tomographic image shooting is to be performed, and the head drive unit 170 is caused to drive to perform the alignment.

When rough alignment is completed, in step S306, the operator performs an operation of pressing a preview start button displayed on the touch panel unit 341. The control terminal 300 that has detected the press of the preview start button causes the shooting control unit 310 to start the two-dimensional scanning of the subject's eye E by the OCTX scanner 153-1 and the OCTY scanner 153-2 and causes the image obtaining unit 330 to sequentially obtain the tomographic image and the eye fundus front image such that the display control unit 350 of the control terminal 300 performs control to display the obtained preview image on the touch panel unit 341 of the display apparatus 301. The state is shifted to a so-called preview state. During this preview, since the eye fundus front image is repeatedly obtained, the eye fundus front images are displayed as video. The eye fundus infrared observation image is also to be obtained. When the eye fundus infrared observation image is obtained, the focus indicator unit 142 is in a state of being arranged on the optical path, and a state is established in which a focus indicator is captured on the eye fundus observation image. In addition, in a preview of the tomographic image, such a setting is made for the tomographic image shooting in S310 which will be described below that overlapping processing is not performed, the shooting position is limited, and the like, and by performing shooting at increased rates of obtaining and displaying the tomographic images, the tomographic images can be obtained in real time.

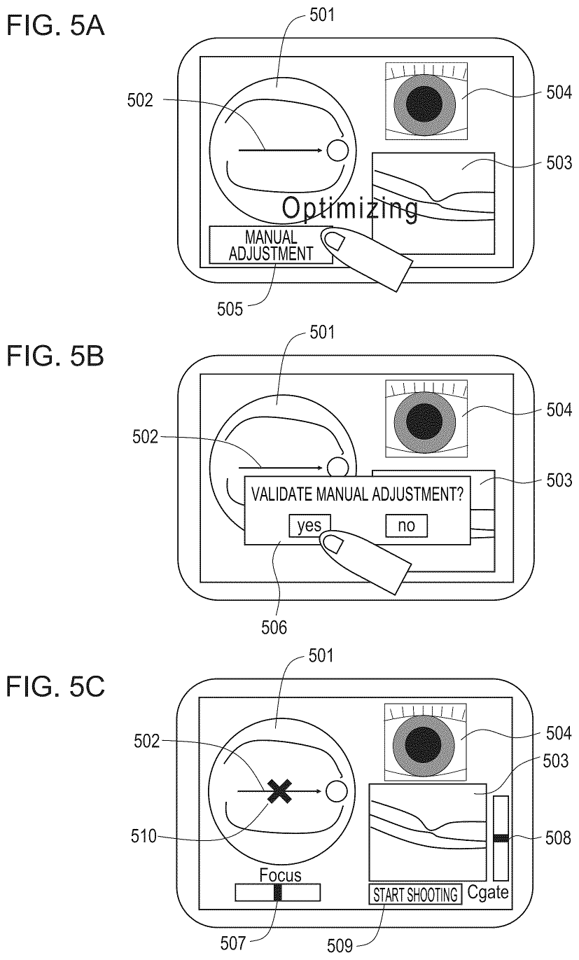

In S307, optimization of the tomographic image is performed. The diopter scale information of the subject's eye is obtained based on the focus indicator captured on the eye fundus infrared observation image, and the shooting control unit 310 executes focus adjustment by way of movements of the focus lens 133 and the focus lens 154 in the optical axis direction in accordance with the diopter scale information. Then, the shooting control unit 310 performs the position adjustment of the reference mirror 160 such that a retinal image is formed on a desired position on the tomographic image based on brightness information of the obtained tomographic image. The image (the eye fundus infrared observation image, the tomographic image at the tomographic image scanning position, or an anterior eye observation image) obtained by the image obtaining unit 330 is displayed by the display control unit 350 on the touch panel unit 341 of the display apparatus 301 as illustrated in FIG. 5A. With this configuration, the operator can check progressions of the focus adjustment and the reference mirror adjustment in real time. It is noted that the focus indicator is often captured on an eye fundus infrared observation image 501, but the focus indicator is not illustrated in FIG. 5A to FIG. 5C.

It is noted that in the tomographic image optimization in S307, a state in which the adjustment is not completed includes a case where the fixation of the subject's eye is not stabilized and the shooting optical axis deviates from the pupil, a case where a pupil diameter of the subject's eye is small and it is difficult to obtain desired focus indicator information, a case where cataracts cause intermediate optic media to become cloudy and it is difficult to obtain the tomographic image with a sufficient brightness, or the like.

In S308, a determination unit (not illustrated) included in the shooting control unit determines whether or not it is necessary to switch the mode to a manual adjustment mode. In a case where the tomographic image optimization in S307 is appropriately completed, it is determined that the tomographic image automatic shooting can be performed, and the flow proceeds to S310 to execute the tomographic image shooting. On the other hand, in a case where the tomographic image optimization in S307 is not appropriately completed, it is determined that the tomographic image automatic shooting is not to be performed, and the mode is switched to the manual adjustment mode.

In the determination on whether or not it is necessary to switch the mode to the manual adjustment mode in the obtainment of the tomographic image in S308 and the determination on whether or not it is necessary to switch the mode to the manual adjustment mode in the obtainment of the eye fundus image in S312 which will be described below, when it is determined that the switching to the manual adjustment mode is not to be performed, the tomographic image shooting in S310 and the eye fundus image shooting in S314 are automatically executed after it is confirmed that the adjustment for the shooting has been correctly performed. For this reason, after the shooting start in S305, a certain length of waiting time period occurs for the operator. During this waiting time period, the display apparatus 301 may be placed on a desk or a display apparatus holder serving as a storage location of the display apparatus 301, and the display apparatus 301 may be carried and moved for a check on an examination conducted in another location. In such a case, an erroneous operation of the display apparatus 301 may occur. For this reason, the control for the switching on the adjustment related to the parameters of the shooting from the auto adjustment mode to the manual adjustment mode may be set as the two-step touch operations including the first touch operation in which the drive instruction related to the type of the drive is to be decided and the second touch operation in which the decided drive instruction is to be validated and transmitted as the drive instruction signal to the control terminal 300.

That is, in the determination on whether or not it is necessary to switch the mode to the manual adjustment mode in S308, when it is determined that the automatic shooting is not to be performed since the optimization (parameter adjustment related to the shooting) is not completed, as illustrated in FIG. 5B, the display control unit 350 performs control to display a screen 506 including a message for requesting a permission to validate the switching to the manual adjustment mode on the touch panel unit 341 of the display apparatus 301. Herein, in a case where the operator performs the operation of selecting Yes, a signal for switching to the manual adjustment mode is transmitted to the control terminal 300, and the shooting control unit 310 switches the mode following a switch signal, so that it becomes possible for the operator to execute the manual adjustment (manual adjustment when the tomographic image is to be obtained) in S309.

In this way, after the first touch operation (press of the Yes button) of deciding that the adjustment of the parameter related to the shooting is to be switched to the adjustment in the manual mode, it also becomes possible to enable execution of various manual adjustment operations (second touch operation).

It is noted that the determination on the switching to the tomographic image manual adjustment mode in S308 is performed by the operator during the execution of the tomographic image optimization in S307, that is, the operator can also perform the determination. At this time, such a configuration may be adopted. That is, the operator may perform the touch operation (first touch operation) on a manual adjustment button 505 displayed on the touch panel unit 341 as illustrated in FIG. 5A to instruct the switching to the manual adjustment mode. As illustrated in FIG. 5B, the screen 506 including a message for requesting a permission to switch the mode to the manual adjustment mode (for validating the manual adjustment) may be displayed, so that the operator may perform the touch operation of selecting Yes (second touch operation) to interrupt automatic shooting, and the mode may be switched to the manual adjustment mode.

In S309, a manual (hand-operated) adjustment of the shooting condition when the tomographic image is to be obtained is performed. The manual adjustment of the shooting condition is performed through an operation on a manual adjustment mode screen displayed on the touch panel unit 341 as illustrated in FIG. 5C. A focus adjustment bar 507 and a reference mirror adjustment bar 508 are displayed on the manual adjustment mode screen. The operator can perform the focus adjustment and the reference mirror adjustment by moving the focus adjustment bar 507 and the reference mirror adjustment bar 508 through the touch operation and the drag operation. In addition, when it is confirmed that the pupil position is deviated on an anterior eye observation image 504, as described above, by performing the touch operation or the drag operation on the position desired to be brought into the center, the head drive unit 170 can be caused to drive.

The example has been explained in which the above-described adjustment operation is performed by only the first touch operation. At this time, for example, such a configuration may be adopted in which pulldown display of the left and right adjustment buttons of the head is carried out by performing the touch operation (first touch operation) on the head adjustment button displayed on the touch panel unit of the display apparatus 301, the adjustment of the left and right position of the head is carried out by performing the touch operation (second touch operation) on the left and right adjustment buttons, and the adjustment is carried out by the first touch operation such as the touch operation or the drag operation of deciding drive instructions (instructions on the drive amount, the drive direction, and the like) on an anterior eye part observation image 504 and the second operation (such as consent to the drive instructions or the like) of transmitting the drive instruction signal.

In addition, the adjustment of the parameter related to the shooting includes, for example, a scan position 502, an internal fixation lamp lighting position 510, and the like which are to be changed by performing a drag operation. Then, after the completion of various adjustments, the operator performs the touch operation (first touch operation) on a shooting start button 509 displayed on the display apparatus 301. At this time, a configuration may be adopted in which the tomographic image shooting in S310 is executed in a case where a screen including a message for requesting a permission to validate shooting start is displayed and the operator performs the operation of selecting Yes (second touch operation for validating the first touch operation).

In S310, the tomographic image shooting is performed. The shooting of the tomographic image is executed, and the desired number of tomographic images with the desired shooting density are obtained to be saved in the storage unit 320. In addition, after the image processing unit 340 implements processing such as noise reduction by overlapping a plurality of images obtained by shooting the same site with one another, the tomographic images can be saved in the storage unit 320.

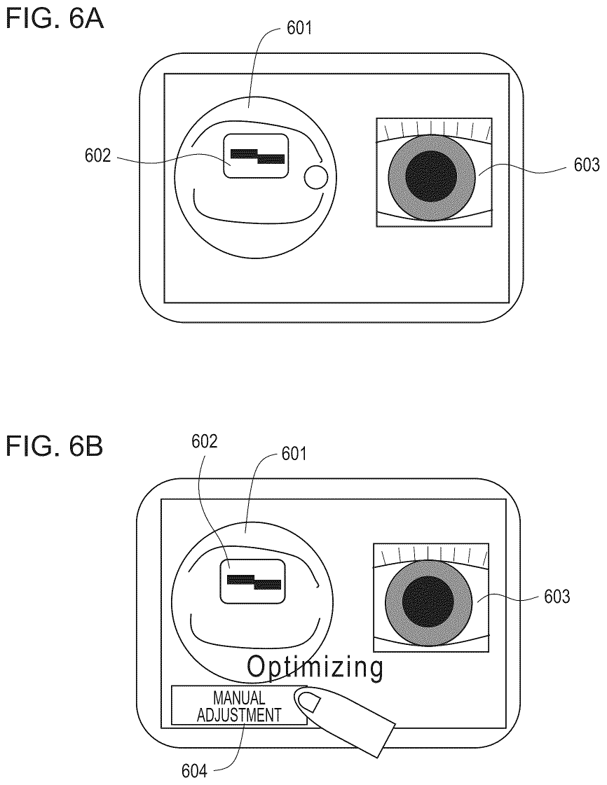

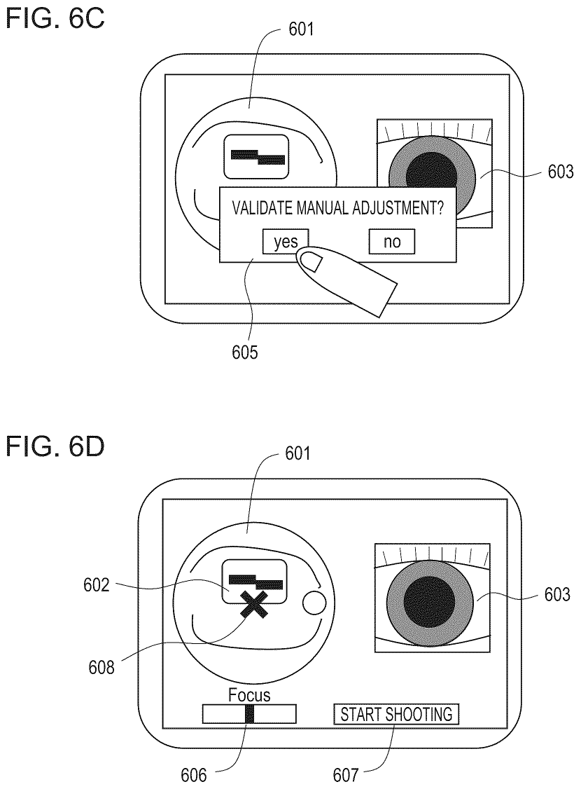

In S311, eye fundus image optimization is executed as a preparation for the eye fundus image shooting. At this time, as illustrated in FIG. 6A, an eye fundus infrared observation image 601, a focus indicator 602, and an anterior eye observation image 603 are displayed on the display screen of the touch panel unit 341 of the display apparatus 301. With this configuration, the operator can check the progressions of the alignment adjustment and the focus adjustment in real time.

In the eye fundus image optimization performed in S311, first, to obtain a positional relationship between the pupil Ep and the optical head unit 100 which is appropriate to the eye fundus image shooting, the shooting control unit 310 causes the head drive unit 170 to drive to execute the alignment. Next, the diopter scale information of the subject's eye is obtained based on the focus indicator 602 captured in the eye fundus infrared observation image 601, and the focus adjustment is executed based on the movement of the focus lens 133 in the optical axis direction in accordance with the diopter scale information. Herein, examples of a case where the focus adjustment becomes necessary include a case where after the focus adjustment is performed through the tomographic image optimization in S307 and the tomographic image manual adjustment in S309, an optical characteristic is changed since the diopter scale adjustment function of the subject's eye is activated, a case where the focus adjustment is performed in a particular site of the tomographic image in the tomographic image manual adjustment in S309 such that a high brightness image is to be obtained, and the like. In addition, it is possible to separately set a focus adjustment condition appropriate to the shooting of the tomographic image and a focus adjustment condition appropriate to the eye fundus image shooting in advance, and in this case too, the focus adjustment needs to be performed again in the eye fundus image optimization in S311.

In S312, the determination is performed on whether or not it is necessary to switch the mode to the manual adjustment mode. In a case where the eye fundus image optimization in S311 is appropriately completed, it is determined that the switching to the manual adjustment mode is not necessary, and the eye fundus image shooting in S314 is executed. On the other hand, in a case where the eye fundus image optimization in S311 is not appropriately completed, it is determined that the switching to the manual adjustment mode is necessary, and the mode is switched to the manual adjustment mode in S313.

It is noted that a state in which the eye fundus image optimization in S311 is not completed occurs in a case where the fixation of the subject's eye is not stabilized and the shooting optical axis deviates from the pupil, a case where the pupil diameter of the subject's eye is small and desired focus indicator information is not to be obtained, or the like.

Upon switching to the manual adjustment mode, similarly as in the switching to the tomographic image manual adjustment mode, a screen 605 including a message for requesting a permission to switch the mode to the manual adjustment mode is displayed as illustrated in FIG. 6C. At this time, a configuration may be adopted in which in a case where the operator performs the touch operation (first touch operation) of selecting Yes, the mode is switched to the manual adjustment mode, and the operator executes the operation (second touch operation) of various manual adjustments in S313.

In addition, the determination on the switching to the manual adjustment mode in S312 can be performed by the instruction from the operator during the execution of the tomographic image optimization in S311 similarly as in the determination on the switching to the tomographic image manual adjustment mode in S308. For example, when the operator performs the touch operation (first touch operation) of pressing a manual adjustment button 604 illustrated in FIG. 6B, the message 605 for requesting the permission to switch the mode to the manual adjustment mode is displayed by the display control unit 350 as illustrated in FIG. 6C. When the operator performs the touch operation (second touch operation) of selecting Yes, the shooting control unit 310 switches the mode to the manual adjustment mode, and it becomes possible for the operator to execute the manual adjustment in S313.

In S313, the manual adjustment of the shooting condition when the eye fundus image is to be obtained is performed. For the manual adjustment of the shooting condition, an operation is performed on the manual adjustment mode screen displayed on the touch panel unit 341 as illustrated in FIG. 6D. The focus adjustment bar 606 is displayed on the manual adjustment mode screen. The operator can perform the focus adjustment by moving the focus adjustment bar 606 through the touch operation.

In addition, when it is confirmed that the pupil position is deviated on the anterior eye observation image 603, the head drive unit 170 can be caused to drive by the touch operation. For example, the head drive unit 170 can be caused to drive by performing the touch operation on the position desired to be brought into the center or the drag operation to the position desired to be brought into the center on the anterior eye observation image 603. It is noted that the drive control of the above-described manual adjustment can also be performed through the first touch operation and the second touch operation as described in S304. In addition, an internal fixation lamp lighting position 608 can be changed by performing the drag operation. Then, after the completion of the various adjustments, the operator performs the touch operation on a shooting start button 607 to perform the eye fundus image shooting. At this time, a configuration may be adopted in which in a case where a screen (not illustrated) which includes a message for requesting a permission to validate the shooting start is displayed and the operator performs an operation of selecting Yes, the drive instruction signal of the ophthalmic shooting apparatus for starting the shooting is transmitted to the ophthalmic shooting apparatus to execute the eye fundus image shooting in S314.

In S314, the eye fundus image shooting is performed. The position of the focus lens 133 is changed to be aligned with the diopter scale position obtained by applying the correction of aberration due to the difference in the wavelengths of the light sources to the diopter scale information of the subject's eye sensed by the infrared LED light source 149 at the same time when the focus indicator unit 142 deviates from the optical path. Then, lighting of the infrared LED light source 149 is stopped, and the white LED light source 150 is caused to emit visible pulsed light so that the eye fundus image is obtained to be saved in the storage unit 320.

In S315, it is determined whether the shot image is satisfactory or unsatisfactory. The display control unit 350 displays the tomographic image shot in the tomographic image shooting in S310 and the eye fundus image shot in the eye fundus image shooting in S314 on a confirmation screen for confirming whether the shot image is satisfactory or unsatisfactory on the touch panel unit 341 of the display apparatus 301, and the operator performs the confirmation on whether the obtained data is satisfactory or unsatisfactory. The confirmation screen is checked, and when it is confirmed that the image desired by the operator has been successfully shot, the touch operation is performed on an OK button displayed on the confirmation screen to end the shooting. On the other hand, in a case where the image desired by the operator has not been successfully shot, an NG button displayed on the confirmation screen is pressed to return to the preview start in S306. Until the image desired by the operator is obtained, the procedure follows the similar flow.

It is noted that the above-described configuration is an example in which after the first touch operation region accepts the first touch operation, the second touch operation region is displayed to perform the second touch operation, but the second touch operation region may be constituted by a plurality of touch operation regions, and an operation of touching the plurality of touch operation regions at the same time as the second touch operation may be performed. For example, this example includes a case where the second touch operation is performed by touching the first touch operation region and a validation button at the same time.

In addition, control of switching the second touch operation region to be hidden may be performed in a case where the second touch operation is not accepted within a predetermined time period since the first touch operation region has accepted the first touch operation to display the second touch operation region. With this configuration, it becomes possible to more effectively avoid occurrence of an erroneous process due to the erroneous touch operation.

In addition, as in the position adjustment of the chin rest and the position adjustment of the optical head part, a configuration may be adopted in which in a case where the adjustment is performed by pressing multiple times a button or position for moving the position displayed on the touch panel unit 341, the drive instructions are issued through the first touch operation and the second touch operation in the operations related to the first position adjustment, and the drive instructions are issued through the single third touch operation which serves both the functions of the operation instructions of the first touch operation and the second touch operation in the operation related to the second position adjustment within a predetermined time period since the operations (the first touch operation and the second touch operation) related to the first position adjustment.

It is noted that according to the present embodiment, the confirmation on whether the shot image is satisfactory or unsatisfactory is performed at the same time for the tomographic image and the eye fundus image, but a flow can be adopted in which each of the images is individually confirmed. In addition, it is possible for the operator to determine and specify how many steps to go back in a case where the image desired by the operator has not been successfully shot.

As described above, by accepting the operations of the drive instructions for changing the relative position of the subject's eye and the ophthalmic shooting apparatus which is controlled by the shooting control unit 310, specifically, the chin rest height adjustment operation in S304, the shooting start operation in S305, the tomographic image manual adjustment in S309, and the position adjustment operation of the head drive unit 170 in the eye fundus image manual adjustment in S313 by way of two-step operations, it becomes possible to avoid the drive related to the shooting due to the erroneous touch operation by the operator (which is the drive to change the relative position of the subject's eye and the ophthalmic shooting apparatus or the like).

### Transformation Example

According to the first embodiment, such a configuration has been adopted in which the display apparatus 301 and the control terminal 300 are separate components which are communicable with each other, and the drive instruction signal related to the ophthalmic shooting apparatus 400 which is generated by the touch operation on the touch panel unit 341 included in the display apparatus 301 is transmitted to the control terminal 300, but as illustrated in FIG. 2B, according to a transformation example, the display apparatus 301 may be a touch panel terminal 301 and may be configured to also serve the function of the control terminal 300.

In the above-described case, the shooting control unit 310 included in the control unit 360 of the touch panel terminal 301 transmits, based on the second touch operation accepted by the touch panel unit 341, a drive control signal for controlling the drive of the ophthalmic shooting apparatus 400 to the ophthalmic shooting apparatus 400, and the head drive unit 170 and the chin rest drive unit 180 of the ophthalmic shooting apparatus 400 perform the drive to change the relative position of the subject and the ophthalmic shooting apparatus based on the drive control signal.

### Second Embodiment

According to the first embodiment, in a case where the display apparatus 301 is grabbed (used as the portable display apparatus) to perform the touch operation or the like, the erroneous drive instruction related to the shooting due to the erroneous touch operation is unlikely to be performed. On the other hand, in a case where the display apparatus 301 is used as a stationary type display apparatus by being fixed in a storage location of the display apparatus 301 such as a holder, the likelihood that the erroneous touch operation occurs becomes sufficiently low. In such a case, when an operation is performed in an erroneous touch avoidance operation mode, the operation for the drive related to the shooting becomes complicated, and it takes time to perform the operation or it is difficult to efficiently perform the operation, which may become rather inconvenient for the operator.

In view of the above, according to a second embodiment, an example of a configuration will be described in which a normal operation mode (in other words, a mode in which the operation for the drive instruction is accepted through the single third touch operation which serves both the functions of the operation instructions of the first touch operation and the second touch operation) that is a first operation mode in which the operation for the drive instruction of changing the relative position of the subject's eye and the ophthalmic shooting apparatus is accepted through the one-step touch operation and the erroneous touch avoidance operation mode that is a second operation mode in which the operation for the drive instruction is accepted through the two-step touch operations can be selected or switched.

When the mode can be switched to the operation mode appropriate to a use situation of the display apparatus in this manner, in a case where the likelihood that the erroneous touch operation occurs is low, the operation can be efficiently performed through the simple operation, and in a case where the likelihood that the erroneous touch operation occurs is high, avoidance of the erroneous operation is prioritized, so that it is possible to make the erroneous drive due to the erroneous touch operation less likely to occur.

With regard to this switching, for example, a configuration may be adopted in which after the login to the shooting software in S300 of FIG. 3, an operation mode selection menu is provided in a setting tab within the patient list screen displayed in the patient selection in S301, and the operator touches the operation mode selection menu to set the operation mode through the operation for the selection between the normal operation mode and the erroneous touch avoidance operation mode. The selected mode, that is, the switch signal is transmitted by the signal transmission unit to the control unit via the signal reception unit. The control unit performs the switching to the selected mode following the switch signal.

According to the second embodiment, the flow is similar to that in S300 to S316 of FIG. 3 except for the selection of the operation mode which is performed in S300 described above. In a case where the normal operation mode is selected, the operation for the drive instruction of changing the relative position of the subject's eye and the ophthalmic shooting apparatus, specifically, the chin rest height adjustment or head position adjustment in S304, the manual adjustment in S309, or the manual adjustment in S313 is performed through the one-step operation. In addition, the shooting start in S305, the tomographic image shooting in S310, and the eye fundus image shooting in S314 may be performed through the one-step operation.

It is noted that with regard to the selection of the operation mode, the explanation has been provided above using the configuration in which the operator selects the operation mode in advance by the operation mode selection menu. However, a configuration may be adopted in which the operator switches the mode in the middle of steps in S300 to S316, or a configuration may be adopted in which the control terminal 300 automatically switches the mode to an erroneous touch avoidance mode based on a signal detected by a sensor included in the display apparatus 301.

In the case of the latter, for example, a configuration may be adopted in which when a touch operation detection sensor included in the display apparatus 301 which is a type of a state detection sensor of the display apparatus 301 detects a touch at the same touched point for a threshold time period or longer, the mode is switched from the normal operation mode to the erroneous touch avoidance mode. A configuration may be adopted in which the display apparatus 301 has state detection sensors of the display apparatus 301 such as an acceleration sensor and a gyro sensor, and when these state detection sensors detect an acceleration and an angle change which are equal to or greater than thresholds, the control terminal 300 switches the mode to the erroneous touch avoidance mode. A configuration may be adopted in which when it is sensed that the state detection sensors are stored in storage locations of the display apparatus (for example, it is sensed that the state detection sensors are stored by sensing that the sensors are connected to power sources or the like), the control terminal 300 switches the mode to the erroneous touch avoidance mode. Of course, the mode may be automatically switched in a case where the display apparatus 301 is fixed to the storage location of the display apparatus 301 such as the holder as described above.

In addition, instead of the decision on whether the operation for the drive instruction of changing the relative position of the subject's eye and the ophthalmic shooting apparatus is to be accepted through the one-step operation or accepted through the two-step operations all uniformly, a plurality of types of drives of changing the relative position of the subject's eye and the ophthalmic shooting apparatus can be prepared, and for each type of the drive, it is possible to set whether the operation is to be accepted through the one-step operation (operation in a normal mode) or accepted through the two-step operations (operation in an erroneous touch avoidance mode).

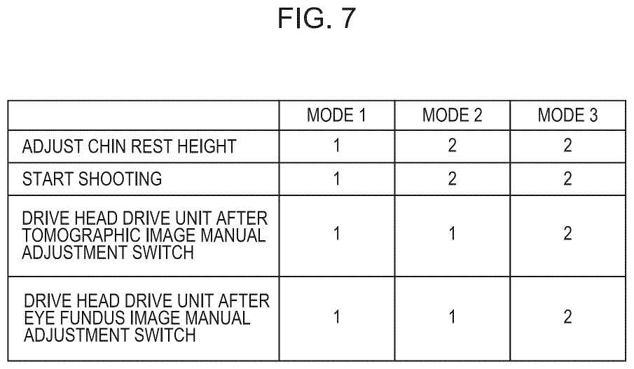

For example, FIG. 7 illustrates the number of necessary operation steps as the operation mode, for example, in a case where three modes are prepared. A mode 1 is a mode in which operations for any of the drive of the head drive unit after the switching of the chin rest height adjustment, the shooting start, and the shooting condition of the tomographic image to the manual adjustment and the drive of the head drive unit after the switching of the shooting condition of the eye fundus image to the manual adjustment are uniformly accepted through the one-step operation. On the other hand, a mode 3 is a mode in which operations for any of the drive of the head drive unit after the switching of the chin rest height adjustment, the shooting start, and the shooting condition of the tomographic image to the manual adjustment and the drive of the head drive unit after the switching of the shooting condition of the eye fundus image to the manual adjustment are all uniformly accepted through the two-step operations. Then, a mode 2 is a mode in which the chin rest height adjustment operation in S304 and the shooting start operation in S305 are accepted through the two-step operations, and the drive of the head drive unit after the switching of the shooting condition of the tomographic image to the manual adjustment and the drive of the head drive unit after the switching of the shooting condition of the eye fundus image to the manual adjustment are accepted through the one-step operation.

As described above, by adopting the configuration in which the operation mode can be selected or switched or the operation mode can be set for each type of the drive, the operation can be set as an optimal operation according to a situation of each operator.

### Third Embodiment

According to the first embodiment, the configuration is adopted in which in the erroneous touch avoidance mode, when the screen is switched on the touch panel unit 341 of the display apparatus 301, the operator is notified that the first touch operation and the second touch operation have been performed. On the other hand, according to a third embodiment, a configuration is adopted in which in the erroneous touch avoidance mode, the operator is notified that the touch operation has been performed by way of sound from a speaker of the display apparatus 301 or vibration caused by an eccentric motor or the like, and with this configuration, the operator finds out that the touch operation has been performed even in a state in which the operator is not looking at the screen of the touch panel unit 341 of the display apparatus 301.

For example, in the chin rest height adjustment in S304 of the first embodiment, when the touch operation (first touch operation) has been performed on a certain single point on the anterior eye part observation image 401 at which the drive instruction of the head drive unit 170 is to be decided is controlled by the control terminal 300 such that the vibration occurs once or the sound such as “whether to perform the chin rest adjustment?” is announced from the speaker. In addition, when the second touch operation is performed in which the operator touches the up and down buttons 404, the vibration continuously occurs twice or the sound such as “the chin rest adjustment has been performed” is announced from the speaker. When such a configuration is adopted, the operator can also recognize from the sound that the operation of changing the relative position of the subject's eye and the ophthalmic shooting apparatus has been performed, and even when an erroneous operation is performed, it is possible to immediately stop the erroneous operation.

In addition, the example has been mentioned in a case where the vibration is to be generated, the vibration is generated once in the first-step operation (first touch operation), and the vibration is continuously generated twice in the second-step operation (second touch operation), but the configuration is not limited to this. Various vibration modes can be prepared in a manner that the first vibration may be set as the short-lasting vibration, and the vibration in the second-step operation may be set as the long-lasting vibration, and the like.

In addition, as in the second embodiment, the display apparatus 301 includes the state detection sensor, and when the state detection sensor detects that the state of the display apparatus 301 being placed in the holder or the like is put into a state of being grabbed, the mode may be switched to a mode in which the vibration is to be generated or the sound is to be announced along with the touch operation.

### Fourth Embodiment

In the medical image shooting system having the erroneous avoidance function of the first embodiment, in a case where the display apparatus 301 or the like is grabbed to perform the touch operation, the drive to change the relative position of the subject's eye and the ophthalmic shooting apparatus due to the erroneous touch operation is less likely to occur. On the other hand, the medical image shooting apparatus which obtains the medical image after the drive to change the relative position of the subject and the medical image shooting apparatus is performed through the touch operation is not limited to the ophthalmic shooting apparatus. The present embodiment is an example in which an X-ray image shooting apparatus is used as the medical image shooting apparatus.

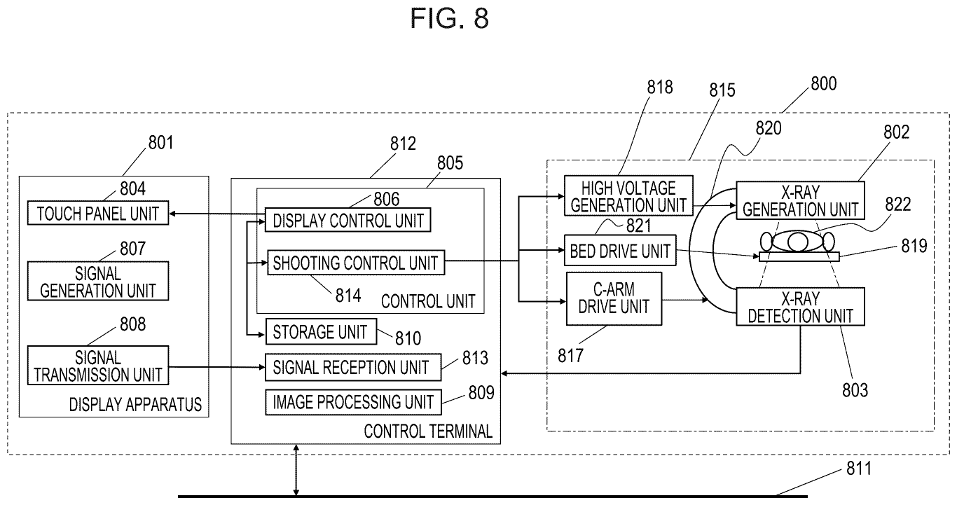

FIG. 8 illustrates a schematic configuration of the X-ray image shooting system of the present embodiment. As illustrated in FIG. 8, an X-ray image shooting system 800 of the present embodiment includes an X-ray image shooting apparatus 815, a control terminal 812, and a display apparatus 801.

The X-ray image shooting apparatus 815 includes an X-ray generation unit 802, an X-ray detection unit 803, a C-arm 820, a C-arm drive unit 817, a high voltage generation unit 818, a bed 819, and a bed drive unit 821. The X-ray generation unit 802, the X-ray detection unit 803, and the C-arm 820 can be collectively referred to as the shooting unit, and the C-arm drive unit 817 can be referred to as the shooting unit drive unit.

The X-ray generation unit 802 includes an X-ray tube, an X-ray filter, and an X-ray aperture device which are not illustrated in the drawing. The X-ray tube is a vacuum tube configured to generate X-ray. The X-ray tube receives application of a high voltage (tube voltage) from the high voltage generation unit 818 and supply of a tube current to generate X-ray from a focal point. The generated X-ray transmits through the X-ray filter, and beam is formed by the X-ray aperture device. The X-ray filter is arranged for a purpose of a reduction in an X-ray exposure amount of the subject, an improvement of an image quality, and the like. For example, the X-ray filter reduces low energy components which are not necessary for the diagnosis with regard to a continuous spectrum of the X-ray generated from the X-ray focal point. The X-ray aperture device limits an X-ray irradiation range following the control of the shooting control unit 814 such that the X-ray which has been generated from the X-ray focal point and transmitted through the X-ray filter is not irradiated outside the shooting range desired by the operator.

The X-ray detection unit 803 includes a plurality of X-ray detection elements. The plurality of X-ray detection elements are arranged in a two-dimensional array-like form. A two-dimensional array-like detector is called a flat panel detector (FPD). Each of the elements in the FPD converts the X-ray which has been generated from the X-ray generation unit 802 and transmitted through a subject 822 into light by a scintillator Layer, and the converted light is detected by the two-dimensional array-like detector. As a result, each of the elements in the FPD outputs an electric signal corresponding to an intensity of the detected X-ray.

The C-arm 820 holds the X-ray generation unit 802 and the X-ray detection unit 803 so as to face each other with the subject 822 therebetween. In addition, the C-arm 820 is driven following the control of the shooting control unit 814 based on the instruction from the operator using the display apparatus 801 which will be described below. When the C-arm drive unit 817 is driven, the relative position of the subject 822 and the X-ray image shooting apparatus can be changed. Accordingly, the alignment between the subject 822 and the X-ray image shooting apparatus can be performed, and the subject 822 can be captured in various directions. Herein, the configuration is also adopted in which an X-ray image shooting unit including the X-ray generation unit 802 and the X-ray detection unit 803 is connected to the C-arm 820 serving as an X-ray image shooting unit drive unit. However, an Q-arm may be used instead of the C-arm 820, or two arms (for example, robot arms or the like) which respectively independently support the X-ray generation unit 802 and the X-ray detection unit 803 so as to face each other may be used. In addition, the C-arm 820 may be configured by a biplane structure having, for example, the C-arm 820 and the Ω-arm.

The bed 819 supports a top panel which is not illustrated in the drawing, and the subject 822 is loaded on the top panel. In the bed 819, the bed drive unit 821 is caused to drive to move the top panel following the control of the shooting control unit 814.

The display apparatus 801 is a portable display apparatus and has a function of displaying an image or the like transmitted from the control terminal 812 and a function as a user interface configured to receive an instruction from the operator as an input. The display apparatus 801 includes a touch panel unit 804 configured to accept an instruction from the operator through a touch operation, a signal generation unit 807 configured to generate a signal related to the instruction accepted by the touch panel unit 804, and a signal transmission unit 808 configured to transmit the generated signal to the control terminal 812.

The control terminal 812 is a terminal configured to control the display apparatus 801, the X-ray generation unit 802, the X-ray detection unit 803, the high voltage generation unit 818, the C-arm drive unit 817, and the bed drive unit 821 and is subjected to network connection as indicated by reference sign 811 to a server which is not illustrated in the drawing. In addition, the control terminal 812 is provided with a control unit 805 including a display control unit 806 and a shooting control unit 814, an image processing unit 809, a storage unit 810, and a signal reception unit 813. The display control unit 806 performs display control on the touch panel unit 804, and the shooting control unit 814 controls each unit related to an X-ray image shooting operation. In addition, the control terminal 812 is connected to each of the high voltage generation unit 818, the X-ray detection unit 803, the C-arm drive unit 817, and the bed drive unit 821 in a wired or wireless manner. It is noted that the display apparatus 801 and the control terminal 812 can be collectively referred to as a control apparatus.

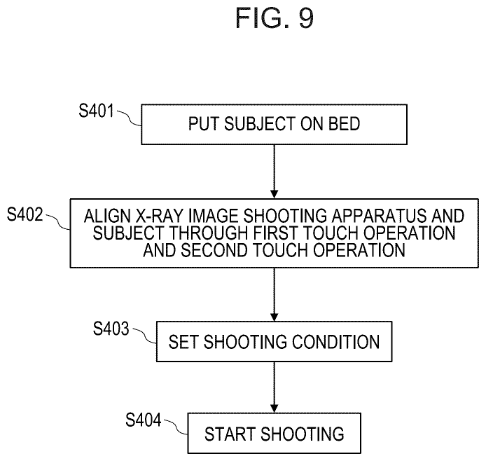

FIG. 9 illustrates a flow of the present embodiment.

As illustrated in FIG. 9, in S401, the subject 822 is put on the bed 819.

In S402, alignment between the subject 822 and the X-ray image shooting apparatus 815 is performed.

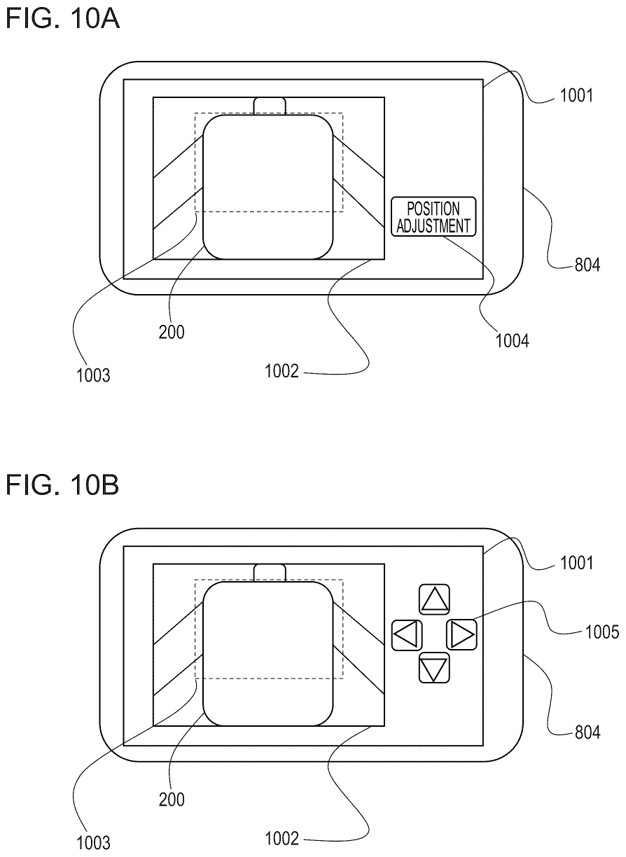

The display control unit 806 displays a position adjustment screen 1001 for adjusting a position of the subject 822 as illustrated in FIG. 10A on the touch panel unit 804. On the position adjustment screen 1001, reference sign 1002 represents a live image which is shot by a camera (not illustrated) attached to the X-ray generation unit 802 in FIG. 8, and reference sign 1003 represents a shooting range to be shot by the X-ray image shooting apparatus.

The operator checks the subject 822 and the shooting range 1003, and in a case where the positions of the subject 822 and the shooting range 1003 are deviated from each other, to adjust a position of the C-arm 820, the first touch operation of pressing a position adjustment button 1004 serving as the first touch operation region included in the position adjustment screen 1001 is performed. Since the first touch operation is a touch operation of enabling the acceptance of the second touch operation, when the touch panel unit 804 accepts the first touch operation, as illustrated in FIG. 10B, the display control unit 806 displays up, down, left, and right buttons 1005 serving as the second touch operation region for performing parameter settings on a drive amount and a direction of the C-arm drive unit 817 on the position adjustment screen 1001. Then, when the operator performs the second touch operation of pressing the up, down, left, and right buttons 1005, according to the number of touches on the up, down, left, and right buttons 1005 and the touch time period, the signal generation unit 807 generates a drive instruction signal with which the C-arm drive unit 817 is driven by a previously set amount. The generated drive instruction signal of the C-arm drive unit 817 is transmitted by the signal transmission unit 808 from the display apparatus 301 to the control terminal 812, and the signal reception unit 813 receives the drive instruction signal, so that the shooting control unit 814 included in the control unit 805 of the control terminal 812 performs control to cause the C-arm drive unit 817 to drive following the drive instruction signal. With this configuration, it becomes possible for the operator to change the relative position of the subject 822 and the X-ray image shooting apparatus 815 and perform the alignment of the shooting range 1003 of the X-ray image shooting apparatus 815 relative to the subject 822. In addition, in the drive of the bed drive unit 821 too, similarly as in the drive control of the C-arm drive unit 817 described above, a configuration may be adopted in which the first touch operation is the touch operation of enabling the acceptance of the second touch operation, and the signal transmission unit 808 transmits the drive instruction signal to the control terminal 812 based on the second touch operation to be received by the signal reception unit 813, so that the bed drive unit 821 is caused to drive to transmit the drive instruction signal for changing the relative position of the subject 822 and the X-ray image shooting apparatus 815 to the control terminal 812.

In S403, the shooting condition and the like are set by using a shooting condition setting screen (not illustrated) displayed on the touch panel unit 804 by the display control unit 806.

In S404, when the operator performs the touch operation of instructing the shooting start on the shooting screen displayed on the touch panel unit 804, the signal generation unit 807 generates a control instruction signal for shooting start. The control instruction signal is transmitted from the signal transmission unit 808 to the control terminal 812 to be received by the signal reception unit 813. Then, the high voltage generation unit 818 is caused to generate a voltage based on the received control instruction signal for the shooting start and also control the X-ray generation unit 802 and the X-ray detection unit 803 to execute the X-ray image shooting.

It is noted that according to the present embodiment, the configuration is adopted in which the display apparatus 801 and the control terminal 812 are the separate components, and the drive instruction signal for the C-arm drive unit 817 and/or the bed drive unit 821 which is generated through the touch operations (the first touch operation and the second touch operation) on the touch panel unit 804 of the display apparatus 801 is transmitted to the control terminal 812, but a configuration may be adopted in which the display apparatus 801 is a touch panel terminal and also serves as the function of the control terminal 812.

### Fifth Embodiment

The first to fourth embodiments correspond to the configuration in which the drive to change the relative position of the subject and the medical image shooting apparatus due to the erroneous touch operation becomes less likely to occur in a case where the display apparatus 301 or the display apparatus 801 is grabbed (used as the portable display apparatus) to perform the touch operation or the like. On the other hand, for example, there is a case where the drive related to the shooting due to the erroneous touch operation is desired to be more strictly avoided such as a case where irradiation of the X-ray to the subject through the erroneous touch operation is desired to be avoided.

In view of the above, according to the present embodiment, a configuration will be described in which the second touch operation is set as a character input operation.

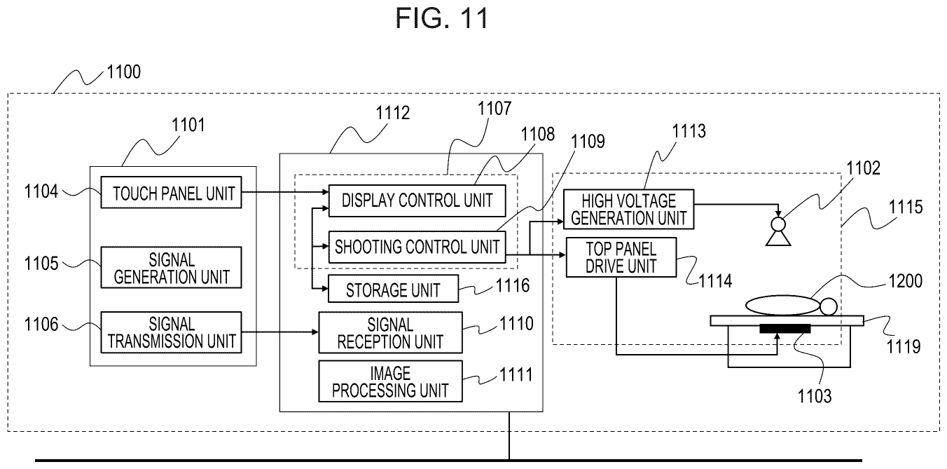

FIG. 11 illustrates a schematic configuration of the X-ray image shooting system of the present embodiment. As illustrated in FIG. 11, an X-ray image shooting system 1100 of the present embodiment includes an X-ray image shooting apparatus 1115, a control terminal 1112, and a display apparatus 1101.

The X-ray image shooting apparatus 1115 includes an X-ray generation unit 1102, an X-ray detection unit 1103, a high voltage generation unit 1113, a top panel 1119, a top panel drive unit 1114 configured to drive the top panel, and a support stand (not illustrated) which supports the top panel.

The control terminal 1112 is provided with a control unit 1107 including a shooting control unit 1109 and a display control unit 1108, a signal reception unit 1110, an image processing unit 1111, and a storage unit 1116.

The display apparatus 1101 includes a touch panel unit 1104, a signal generation unit 1105, and a signal transmission unit 1106. It is noted that with regard to each of components constituting the X-ray image shooting apparatus 1115, the control terminal 1112, and the display apparatus 1101, parts having the same phrases as those in the fourth embodiment are set to have the similar functions.

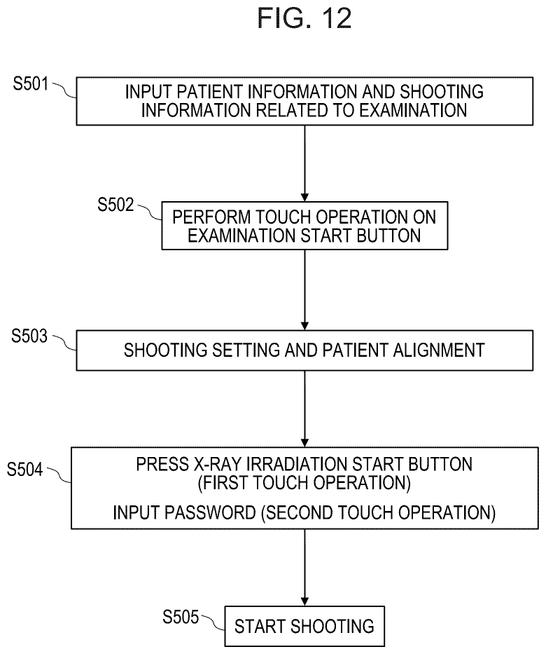

FIG. 12 illustrates the shooting flow according to the present embodiment.

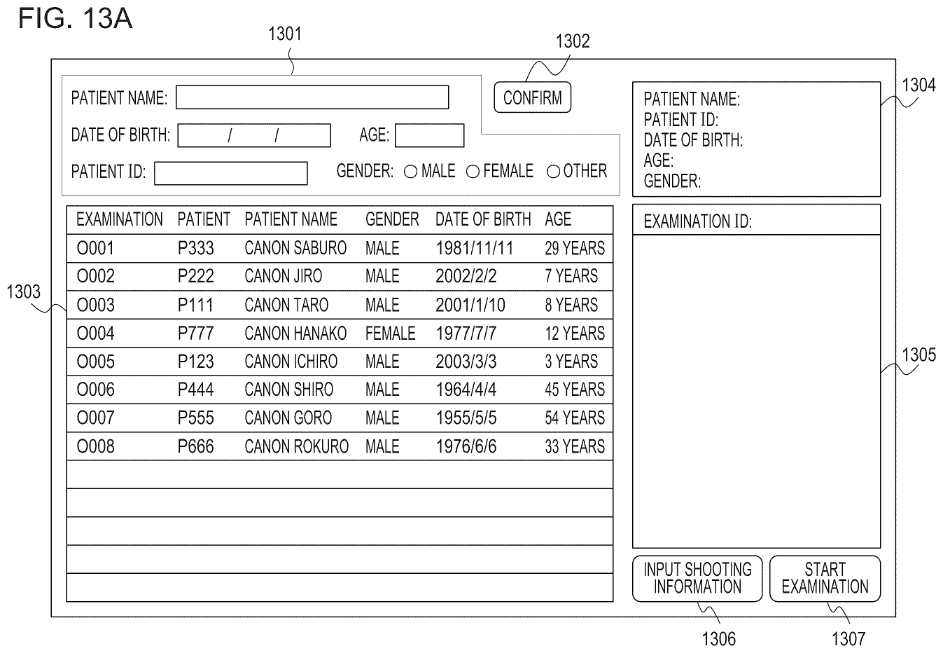

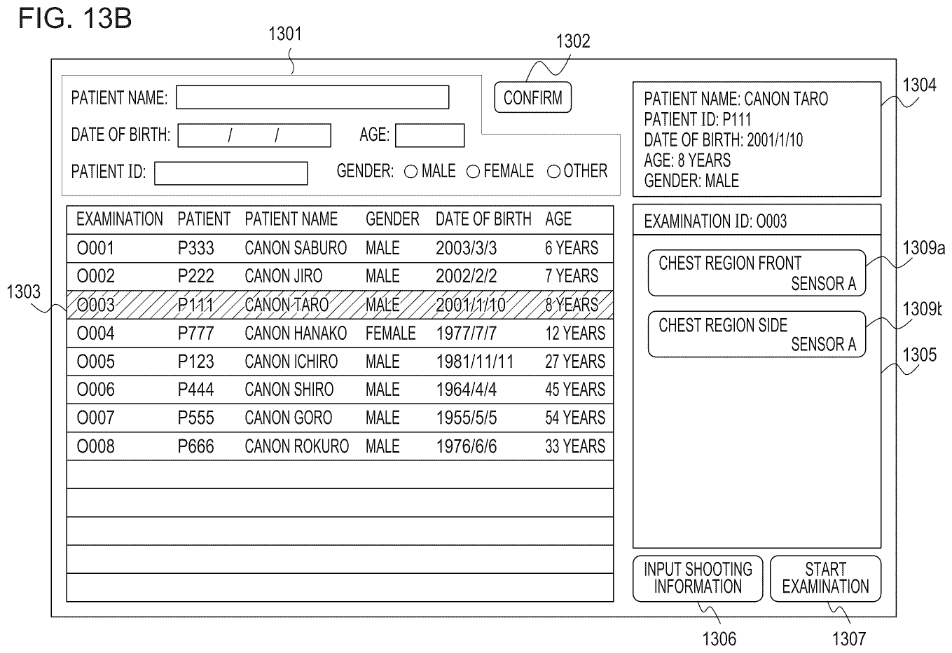

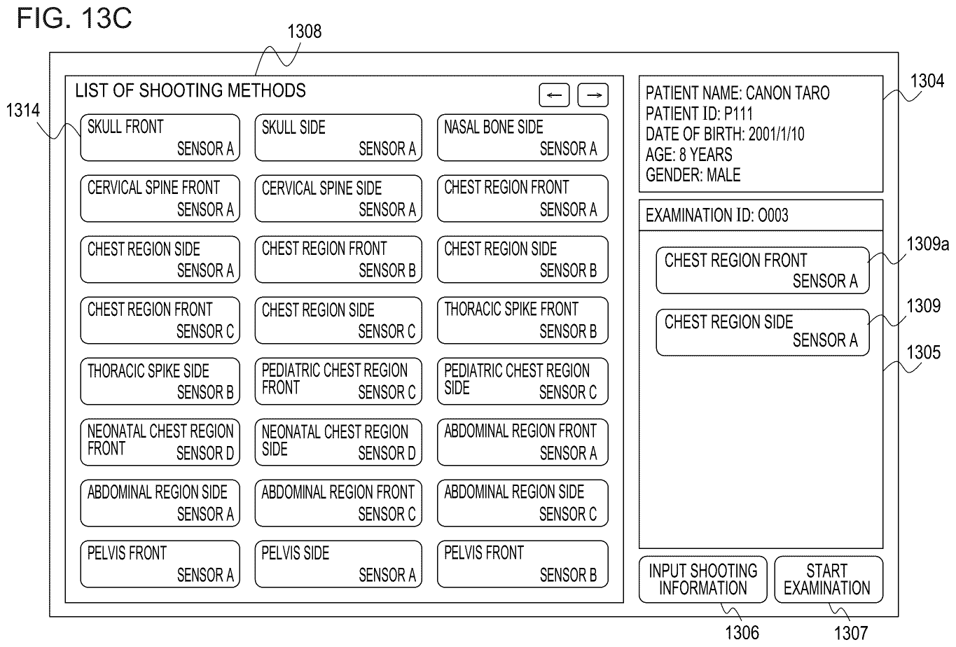

In S501, as denoted by reference signs 1301 and 1302, the operator (also referred to as an examiner) inputs patient information and examination information to the display apparatus 1101 based on an examination request form or the like. As illustrated in FIG. 13A, a new examination input screen is displayed on the touch panel unit 1104 included in the display apparatus 1101 by the display control unit 1108 of the control terminal 1112. The new examination input screen is constituted by including a patient information display region 1304, a shooting information display region 1305, a shooting information input button 1306, and an examination start button 1307.

Examinations received by the server which is not illustrated in the drawing are itemized in a requested examination list 1303 to realize display in a list. When any of the examinations is selected through the touch operation from the requested examination list 1303, as illustrated in FIG. 13B, patient information (such as a patient ID, a patient name, and a date of birth) corresponding to the selected patient is displayed in the patient information display region 1304.

In addition, an examination ID is displayed in the shooting information display region 1305, and shooting information corresponding to the examination ID is displayed in a region below the shooting information display region 1305. The shooting information is received via a network from another information processing apparatus or the like, for example. In the case of the example in FIG. 13B, shooting method buttons 1309 (a chest region front button 1309a and a chest region side button 1309b) corresponding to the shooting information are displayed. When the operator performs the touch operation on the shooting information input button 1306, a shooting information input region 1308 is displayed as illustrated in FIG. 13C, and it is also possible to further add a shooting method. In the case of the example FIG. 13C, a plurality of shooting method selection buttons 1314 are displayed in the shooting information input region 1308, and a shooting method can be added by performing the touch operation of selecting this shooting method. The added shooting method is displayed in the shooting information display region 1305 along with the chest region front button 1309a and the chest region side button 1309b. Each shooting method is associated with a shooting method ID.

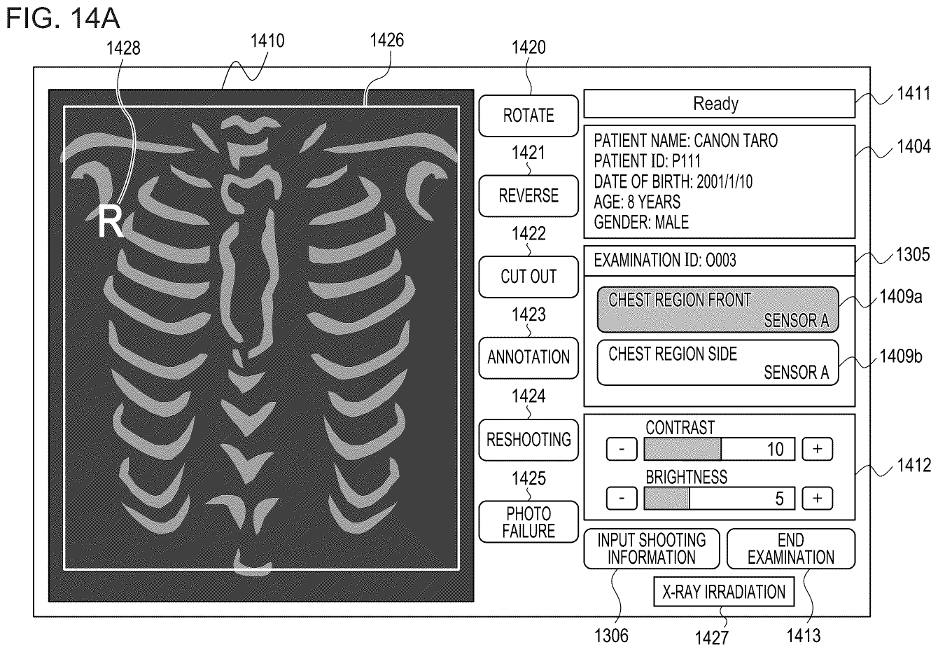

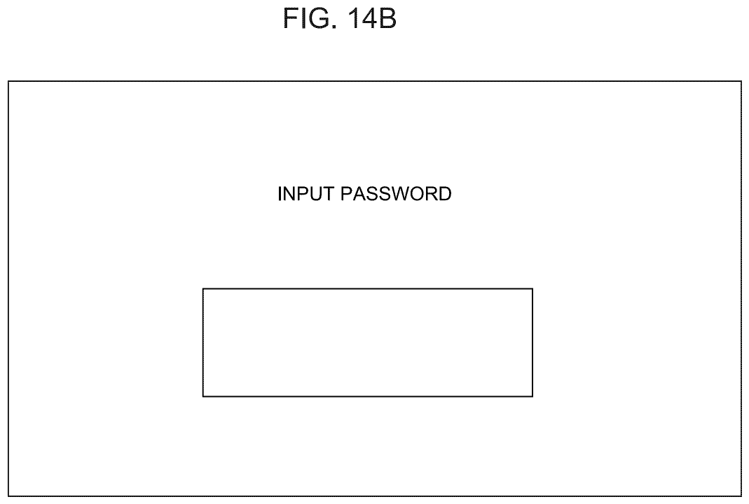

In S502, after the patient information and the shooting information have been checked, the operator performs the touch operation on the examination start button 1307. With this configuration, an examination to be implemented is confirmed, and a preparation for the examination is started. Following the touch operation on the examination start button 1307, the control unit 1107 for display is to display a shooting screen as illustrated in FIG. 14A on the touch panel unit 1104. The shooting screen is a screen used upon shooting.

Basically, the shooting screen is constituted by having the display region similar to the new examination input screen described with reference to FIG. 13A to FIG. 13C. As also denoted by reference signs 1404 to 1428, examples of display regions to be newly added include, as illustrated in FIG. 14A, an image display region 1410, a message region 1411, an image processing setting region 1412, and an examination end button 1413.

When the shooting screen is displayed, a shooting method button 1409a arranged at an uppermost part in the shooting information display region 1305 is in a selected state by default. According to this, the shooting control unit 1109 included in the control unit 1107 provided to the control terminal 1112 controls the high voltage generation unit 1113 and the X-ray detection unit 1103 following the shooting conditions (such as a tube voltage, a tube current, and an irradiation time) which have been set corresponding to the shooting method button (shooting method), and the preparation is to be completed.

When the preparation is completed, the X-ray image shooting system shifts to a shooting ready state. At this time, a “ready message” indicating that the shooting can be performed is displayed in the message region 1411.

In S503, the operator confirms the shooting method to perform the setting for the shooting and the alignment of the subject 1200. At this time, similarly as in the fourth embodiment, the alignment between the X-ray image shooting apparatus 1115 and the subject 1200 (drive of the top panel drive unit 1114) may be performed through the first touch operation of enabling the acceptance of the second touch operation and the second touch operation.

In S504, the operator completes the series of shooting preparations to refer to the message region 1411, and after it is confirmed that the state is the shooting ready state (ready), and the operator performs the first touch operation that is the touch operation of pressing an X-ray irradiation button 1427. When the first touch operation is performed, the display control unit 1108 displays a screen for inputting a password as exemplified in FIG. 14B on the touch panel unit 1104. Then, when the operator inputs the password on the screen, the control unit 1107 performs collation between the input character and the previously set character. In a case where a collation result indicates the match, the flow proceeds to S505. In a case where the collation result indicates no match, the display control unit 1108 displays a message indicating no match on the touch panel unit 1104, and displays the screen for inputting a password again. It is noted that the password to be input on the second touch operation may be a character displayed on the touch panel unit 1104 after the first touch operation. In addition, herein, the second touch operation is set as the password input operation, but any character input operation may be adopted.

In S505, in response to the match between the character input in S504 and a previously set password, the shooting control unit 1109 causes the high voltage generation unit 1113 to perform application of a voltage to transmit a control signal for causing the X-ray generation unit 1102 to generate X-ray. The X-ray generation unit 1102 irradiates the X-ray towards the subject (particular site of the patient) 1200 based on the control signal. Then, the X-ray detection unit 1103 detects the X-ray which has transmitted through the subject 1200, and the image processing unit 1111 generates an image based on the detected signal to be stored in the storage unit 1116, and displays the shot image on the touch panel unit 1104 of the display apparatus 1101.

It is noted that according to the present embodiment, the configuration is adopted in which the display apparatus 1101 and the control terminal 1112 are separate components, and a control signal related to irradiation start of the X-ray generated through the touch operation on the touch panel unit 1104 of the display apparatus 1101 is transmitted to the control terminal 1112, but similarly as in the transformation example, a configuration may be adopted in which the display apparatus 1101 is a touch panel terminal and also serves the functions of the control terminal 1112.

In addition, according to the present embodiment, the configuration is adopted in which based on the second touch operation, the X-ray generation unit 1102 transmits an instruction signal for irradiating X-ray to the control terminal 1112, but any signal for the instruction related to the shooting may be used as the signal to be transmitted to the control terminal 1112 based on the second touch operation. For example, the drive instruction signal for the shooting start or the drive instruction signal for changing the relative position of the subject and the medical image shooting apparatus as in the first to fourth embodiments may be used.

### Other Embodiment

The present invention is not limited to the above-described embodiments and can be implemented by way of various modifications and alterations in a scope without departing from the gist of the present invention. The embodiments have been explained using the cases as examples in which according to the first to third embodiments described above, the ophthalmic shooting apparatus which performs the tomographic image shooting and the eye fundus image shooting is used as the medical image shooting apparatus that shoots the subject, and according to the fourth and fifth embodiments, the X-ray image shooting apparatus is used as the medical image shooting apparatus that shoots the subject. However, the medical image shooting apparatus according to the present invention is not limited to this. For example, the embodiments of the present invention can be applied to an X-ray image shooting apparatus that shoots a medical image by exposing with X-ray to a subject, such as an apparatus configured to obtain a CT image, a dual energy CT image, or a photon-counting computed tomography (PCCT) image, or a shooting apparatus which obtains a medical image by exposing the subject to an electromagnetic wave, such as an apparatus configured to obtain a magnetic resonance imaging (MRI) image. In addition, in a case where the medical image shooting apparatus is an ophthalmic shooting apparatus, an ophthalmic shooting apparatus for a purpose of shooting any one of or at least one of the tomographic image and eye fundus image shooting may be adopted, or another model of the ophthalmic shooting apparatus such as a refractometer configured to measure refractive power of the eye may be adopted. In addition, orders of the shooting flow may be switched.

In addition, the present invention includes a case where by executing a medical image shooting program code read by a computer, an operating system (OS) or the like running on the computer performs part or all of actual processing, so that the function of the embodiment described above is realized through the processing.

Furthermore, the present invention includes a case where a program code read from a recording medium is written to a memory in a function expansion card or a function expansion unit attached to the computer, and a computing apparatus in the function expansion card or the function expansion unit performs part or all of actual processing, so that the function of the embodiment described above is realized through the processing.

In a case where the present invention is applied to the recording medium, program codes corresponding to the drawings described above are stored in the recording medium.

With respect to the above embodiments, the following appendixes are disclosed as an aspect and optional features of the invention.

### Appendix 1

A touch panel terminal which is capable of communicating with a medical image shooting apparatus that shoots a subject and which is portable, includes:
a touch panel unit configured to perform display for a touch operation and accept a touch operation;
a control unit configured to generate a drive instruction signal for instructing drive of the medical image shooting apparatus based on the touch operation accepted by the touch panel unit; and
a signal transmission unit configured to transmit the drive instruction signal to the medical image shooting apparatus, in which
the touch panel unit accepts a first touch operation and a second touch operation,
the first touch operation is a touch operation of enabling an acceptance of the second touch operation, and
the signal transmission unit transmits, based on the second touch operation, a drive instruction signal for changing a relative position of the subject and the medical image shooting apparatus to the medical image shooting apparatus.

### Appendix 2

The touch panel unit may include a first touch panel region in which the first touch operation is accepted and a second touch panel region in which the second touch operation is accepted.

### Appendix 3

The control unit may include a display control unit configured to perform display control on the touch panel unit, and
the display control unit may perform control to display the second touch panel region on the touch panel unit when the first touch panel region displayed on the touch panel unit accepts the first touch operation.

### Appendix 4

The control unit may perform control to hide the second touch panel region in a case where the second touch operation is not accepted within a predetermined time period since the second touch panel region is displayed.

### Appendix 5

The second touch panel region may be constituted by a plurality of touch panel regions.

### Appendix 6

The plurality of touch panel regions may include the first touch panel region.

### Appendix 7

The control unit may perform control to generate sound or vibration when the touch panel unit accepts the first touch operation or the second touch operation.

### Appendix 8

The second touch operation may be an operation on the second touch panel region which is continuous to the first touch operation.

### Appendix 9

The touch panel unit may have a first operation mode in which the signal transmission unit transmits the drive instruction signal to the medical image shooting apparatus by accepting both operations of the first touch operation and the second touch operation and a second operation mode in which the signal transmission unit transmits the drive instruction signal to the medical image shooting apparatus by accepting a single third touch operation which serves both functions of operation instructions of the first touch operation and the second touch operation.

### Appendix 10

The drive instruction signal may be a drive instruction signal related to a plurality of types of drives of the medical image shooting apparatus, and
at least one drive among the plurality of types of drives may be drive in the first operation mode, and at least one other drive may be drive in the second operation mode.

### Appendix 11

The control unit may perform control for switching from the second operation mode to the first operation mode when the touch panel region accepts an operation for the switching from the second operation mode to the first operation mode.

### Appendix 12

The control unit may perform control for automatic switching from the second operation mode to the first operation mode.

### Appendix 13

The control for the switching from the second operation mode to the first operation mode may be performed by the control unit based on a signal detected by a state detection sensor of the touch panel terminal which is included in the touch panel terminal.

### Appendix 14

The state detection sensor may be a sensor configured to detect an angle or an acceleration of the touch panel terminal, and in a case where a signal related to the angle or the acceleration of the touch panel terminal which is detected by the state detection sensor exceeds a predetermined range, the control unit may perform the control for the switching from the second operation mode to the first operation mode.

### Appendix 15

The state detection sensor may be a sensor configured to detect a position of a touched point to the touch panel terminal and a touched time period, and in a case where a continuous touch operation at a same touched point to the touch panel terminal is detected for longer than a predetermined time period, the control unit may perform the control for the switching from the second operation mode to the first operation mode.

### Appendix 16

The state detection sensor may be a sensor configured to sense that the touch panel terminal is stored in a storage location of the touch panel terminal, and when the state detection sensor senses that the touch panel terminal is stored, the control unit may perform the control for the switching from the second operation mode to the first operation mode.

### Appendix 17

A medical image shooting apparatus may be configured which includes:
a signal reception unit capable of receiving a drive instruction signal related to shooting of the medical image shooting apparatus from the touch panel terminal;
a drive control signal generation unit configured to generate a drive control signal related to the shooting of the medical image shooting apparatus based on the drive instruction signal;
a drive unit configured to perform drive of the medical image shooting apparatus based on the drive control signal; and
a shooting unit configured to shoot a subject after the drive is performed.

### Appendix 18

The medical image shooting apparatus may be an ophthalmic shooting apparatus.

### Appendix 19

A medical image shooting program for a touch panel terminal which is capable of communicating with a medical image shooting apparatus that shoots a subject and which is portable, the medical image shooting program including:
accepting, by a touch panel unit included in the touch panel terminal and configured to perform display for a touch operation and accept a touch operation, a first touch operation of enabling an acceptance of a second touch operation and the second touch operation; and
transmitting a drive instruction signal for changing a relative position of the subject and the medical image shooting apparatus to the medical image shooting apparatus based on the second touch operation.

In addition, with regard to the above-described embodiments, the following configurations will also be disclosed as one aspect and optional features of the invention.

### Configuration 1

A control apparatus for a medical image shooting apparatus is a control apparatus for a medical image shooting apparatus including a control terminal configured to control a medical image shooting apparatus that shoots a subject and a display apparatus which is capable of communicating with the control terminal and which is portable, in which
the display apparatus includes
a touch panel unit configured to perform display for a touch operation and accept a touch operation,
a signal generation unit configured to generate a drive instruction signal for instructing drive of a drive unit included in the medical image shooting apparatus based on the touch operation accepted by the touch panel unit, and
a signal transmission unit configured to transmit the drive instruction signal to the control terminal that controls the medical image shooting apparatus,
the control terminal includes
a signal reception unit configured to receive the drive instruction signal, and
a control unit configured to perform control to cause the drive unit of the medical image shooting apparatus to drive based on the drive instruction signal received by the signal reception unit,
the touch panel unit accepts a first touch operation and a second touch operation,
the first touch operation is a touch operation of enabling an acceptance of the second touch operation, and
the signal transmission unit transmits, based on the second touch operation accepted by the touch panel unit, a drive instruction signal for changing a relative position of the subject and the medical image shooting apparatus to the control terminal.

### Configuration 2

The control apparatus for the medical image shooting apparatus according to Configuration 1, in which the control terminal includes
a display control unit configured to display, on the touch panel unit, a first touch operation region in which the first touch operation is accepted and a second touch operation region in which the second touch operation is accepted.

### Configuration 3

The control apparatus for the medical image shooting apparatus according to Configuration 2, in which the display control unit performs control to display the second touch operation region on the touch panel unit when the first touch operation region displayed on the touch panel unit accepts the first touch operation.

### Configuration 4

The control apparatus for the medical image shooting apparatus according to Configuration 2 or 3, in which the display control unit performs control to hide the second touch operation region in a case where the second touch operation is not accepted within a predetermined time period since the second touch operation region is displayed.

### Configuration 5

The control apparatus for the medical image shooting apparatus according to any one of Configurations 2 to 4, in which the second touch operation region is constituted by a plurality of touch operation regions.

### Configuration 6

The control apparatus for the medical image shooting apparatus according to Configuration 5, in which the plurality of touch operation regions include the first touch operation region.

### Configuration 7

The control apparatus for the medical image shooting apparatus according to any one of Configurations 1 to 6, in which the control unit performs control to generate sound or vibration when the touch panel unit accepts the first touch operation or the second touch operation.

### Configuration 8

The control apparatus for the medical image shooting apparatus according to any one of Configurations 2 to 7, in which the second touch operation is an operation on the second touch operation region which is continuous to the first touch operation.

### Configuration 9

The control apparatus for the medical image shooting apparatus according to any one of Configurations 1 to 8, in which the touch panel unit has a first operation mode in which the signal transmission unit transmits the drive instruction signal to the control apparatus for the medical image shooting apparatus by accepting both operations of the first touch operation and the second touch operation and a second operation mode in which the signal transmission unit transmits the drive instruction signal to the control apparatus for the medical image shooting apparatus by accepting a single third touch operation which serves both functions of operation instructions of the first touch operation and the second touch operation.

### Configuration 10

The control apparatus for the medical image shooting apparatus according to Configuration 9, in which
the drive instruction signal is a drive instruction signal related to a plurality of types of drives of the medical image shooting apparatus, and
at least one drive among the plurality of types of drives is drive in the first operation mode, and at least one other drive is drive in the second operation mode.

### Configuration 11

The control apparatus for the medical image shooting apparatus according to Configuration 8 or 9, in which when the touch panel unit accepts an operation for switching from the second operation mode to the first operation mode, the control unit performs control for the switching from the second operation mode to the first operation mode.

### Configuration 12

The control apparatus for the medical image shooting apparatus according to Configuration 9 or 10, in which the control unit performs control for automatic switching from the second operation mode to the first operation mode.

### Configuration 13

The control apparatus for the medical image shooting apparatus according to Configuration 12, in which the control for the switching from the second operation mode to the first operation mode is performed by the control unit based on a signal detected by a state detection sensor of the display apparatus which is included in the display apparatus.

### Configuration 14

The control apparatus for the medical image shooting apparatus according to Configuration 13, in which the state detection sensor is a sensor configured to detect an angle or an acceleration of the display apparatus, and in a case where a signal related to the angle or the acceleration of the display apparatus which is detected by the state detection sensor exceeds a predetermined range, the control unit performs the control for the switching from the second operation mode to the first operation mode.

### Configuration 15

The control apparatus for the medical image shooting apparatus according to Configuration 13, in which the state detection sensor is a sensor configured to detect a position of a touched point to the touch panel unit and a touched time period, and in a case where a continuous touch operation at a same touched point to the display apparatus is detected for longer than a predetermined time period, the control unit performs the control for the switching from the second operation mode to the first operation mode.

### Configuration 16

The control apparatus for the medical image shooting apparatus according to Configuration 13, in which the state detection sensor is a sensor configured to sense that the display apparatus is stored in a storage location of the display apparatus, and when the state detection sensor senses that the display apparatus is stored, the control unit performs the control for the switching from the second operation mode to the first operation mode.

### Configuration 17

The control apparatus for the medical image shooting apparatus according to any one of Configurations 1 to 16, in which the second touch operation is an operation of inputting a previously set character.

### Configuration 18

The control apparatus for the medical image shooting apparatus according to any one of Configurations 1 to 17, in which the medical image shooting apparatus is an ophthalmic shooting apparatus.

### Configuration 19

A touch panel terminal which is capable of communicating with a medical image shooting apparatus that shoots a subject and which is portable,
the touch panel terminal including:
a touch panel unit configured to perform display for a touch operation and accept a touch operation;
a control unit configured to generate a drive control signal for controlling drive of the medical image shooting apparatus based on the touch operation accepted by the touch panel unit; and
a signal transmission unit configured to transmit the drive control signal to the medical image shooting apparatus, in which
the touch panel unit accepts a first touch operation and a second touch operation,
the first touch operation is a touch operation of enabling an acceptance of the second touch operation, and
the signal transmission unit transmits, based on the second touch operation, a drive control signal for changing a relative position of the subject and the medical image shooting apparatus to the medical image shooting apparatus.

### Configuration 20

A medical image shooting system including:
a medical image shooting apparatus; and
a control apparatus configured to control the medical image shooting apparatus, in which
the control apparatus for the medical image shooting apparatus is the control apparatus for the medical image shooting apparatus according to any one of Configurations 1 to 18.

### Configuration 21

A control apparatus for a medical image shooting apparatus including a control terminal configured to control a medical image shooting apparatus that shoots a subject and a display apparatus which is capable of communicating with the control terminal and which is portable, in which
the display apparatus includes a touch panel unit configured to perform display for a touch operation and accept a touch operation, a signal generation unit configured to generate a control instruction signal related to shooting of the medical image shooting apparatus based on the touch operation accepted by the touch panel unit, and a signal transmission unit configured to transmit the control instruction signal to the control terminal that controls the
the control terminal includes
a signal reception unit configured to receive the control instruction signal, and
a control unit configured to perform control related to the shooting of the medical image shooting apparatus based on the received control instruction signal,
the touch panel unit accepts a first touch operation and a second touch operation,
the first touch operation is a touch operation of enabling an acceptance of the second touch operation,
the second touch operation is a character input operation, and
the control unit performs the control related to the shooting of the medical image shooting apparatus based on the second touch operation accepted by the touch panel unit.

### Configuration 22

The control apparatus for the medical image shooting apparatus according to Configuration 21, in which the medical image shooting apparatus is an X-ray image shooting apparatus, and the control related to the shooting is control of starting X-ray irradiation.

### Program 1

A control program for a medical image shooting apparatus that shoots a subject, the control program including:
accepting, by a touch panel unit which is provided to a portable display apparatus capable of communicating with a control terminal that controls the medical image shooting apparatus and which is configured to perform display for a touch operation and accept a touch operation, a first touch operation of enabling an acceptance of a second touch operation and the second touch operation; and
transmitting a drive instruction signal for changing a relative position of the subject and the medical image shooting apparatus to the medical image shooting apparatus based on the second touch operation.

### Program 2

A control program for a medical image shooting apparatus that shoots a subject, the control program including:
accepting, by a touch panel unit which is provided to a portable display apparatus capable of communicating with a control terminal that controls the medical image shooting apparatus and which is configured to perform display for a touch operation and accept a touch operation, a first touch operation of enabling an acceptance of a second touch operation and the second touch operation; and
performing, by a control unit provided to the control terminal and configured to control the medical image shooting apparatus, control to cause a drive unit of the medical image shooting apparatus to perform drive related to shooting based on the second touch operation, in which
the second touch operation is a character input operation.

According to the embodiments of the present invention, it is possible to provide the control apparatus for the medical image shooting apparatus in which the erroneous drive of the medical image shooting apparatus due to the erroneous touch operation hardly occurs, the touch panel terminal, the medical image shooting system, and the control program for the medical image shooting apparatus.

### OTHER EMBODIMENTS

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. A control apparatus for a medical image shooting apparatus, comprising:
a control terminal configured to control a medical image shooting apparatus that shoots a subject; and
a display apparatus which is capable of communicating with the control terminal and which is portable, wherein
the display apparatus includes
a touch panel unit configured to perform display for a touch operation and accept a touch operation,
a signal generation unit configured to generate a drive instruction signal for instructing drive of a drive unit included in the medical image shooting apparatus based on the touch operation accepted by the touch panel unit, and
a signal transmission unit configured to transmit the drive instruction signal to the control terminal that controls the medical image shooting apparatus,
the control terminal includes
a signal reception unit configured to receive the drive instruction signal, and
a control unit configured to perform control to cause the drive unit of the medical image shooting apparatus to drive based on the drive instruction signal received by the signal reception unit,
the touch panel unit accepts a first touch operation and a second touch operation,
the first touch operation is a touch operation of enabling an acceptance of the second touch operation, and
the signal transmission unit transmits, based on the second touch operation accepted by the touch panel unit, a drive instruction signal for changing a relative position of the subject and the medical image shooting apparatus to the control terminal.

2. The control apparatus for the medical image shooting apparatus according to claim 1, wherein the control terminal includes
a display control unit configured to display, on the touch panel unit, a first touch operation region in which the first touch operation is accepted and a second touch operation region in which the second touch operation is accepted.

3. The control apparatus for the medical image shooting apparatus according to claim 2, wherein the display control unit performs control to display the second touch operation region on the touch panel unit when the first touch operation region displayed on the touch panel unit accepts the first touch operation.

4. The control apparatus for the medical image shooting apparatus according to claim 2, wherein the display control unit performs control to hide the second touch operation region in a case where the second touch operation is not accepted within a predetermined time period since the second touch operation region is displayed.

5. The control apparatus for the medical image shooting apparatus according to claim 2, wherein the second touch operation region is constituted by a plurality of touch operation regions.

6. The control apparatus for the medical image shooting apparatus according to claim 5, wherein the plurality of touch operation regions include the first touch operation region.

7. The control apparatus for the medical image shooting apparatus according to claim 1, wherein the control unit performs control to generate sound or vibration when the touch panel unit accepts the first touch operation or the second touch operation.

8. The control apparatus for the medical image shooting apparatus according to claim 2, wherein the second touch operation is an operation on the second touch operation region which is continuous to the first touch operation.

9. The control apparatus for the medical image shooting apparatus according to claim 1, wherein the touch panel unit has a first operation mode in which the signal transmission unit transmits the drive instruction signal to the control apparatus for the medical image shooting apparatus by accepting both operations of the first touch operation and the second touch operation and a second operation mode in which the signal transmission unit transmits the drive instruction signal to the control apparatus for the medical image shooting apparatus by accepting a single third touch operation which serves both functions of operation instructions of the first touch operation and the second touch operation.

10. The control apparatus for the medical image shooting apparatus according to claim 9, wherein the drive instruction signal is a drive instruction signal related to a plurality of types of drives of the medical image shooting apparatus, and
at least one drive among the plurality of types of drives is drive in the first operation mode, and at least one other drive is drive in the second operation mode.

11. The control apparatus for the medical image shooting apparatus according to claim 8, wherein when the touch panel unit accepts an operation for switching from the second operation mode to the first operation mode, the control unit performs control for the switching from the second operation mode to the first operation mode.

12. The control apparatus for the medical image shooting apparatus according to claim 9, wherein the control unit performs control for automatic switching from the second operation mode to the first operation mode.

13. The control apparatus for the medical image shooting apparatus according to claim 12, wherein the control for the switching from the second operation mode to the first operation mode is performed by the control unit based on a signal detected by a state detection sensor of the display apparatus which is included in the display apparatus.

14. The control apparatus for the medical image shooting apparatus according to claim 13, wherein the state detection sensor is a sensor configured to detect an angle or an acceleration of the display apparatus, and in a case where a signal related to the angle or the acceleration of the display apparatus which is detected by the state detection sensor exceeds a predetermined range, the control unit performs the control for the switching from the second operation mode to the first operation mode.

15. The control apparatus for the medical image shooting apparatus according to claim 13, wherein the state detection sensor is a sensor configured to detect a position of a touched point to the touch panel unit and a touched time period, and in a case where a continuous touch operation at a same touched point to the display apparatus is detected for longer than a predetermined time period, the control unit performs the control for the switching from the second operation mode to the first operation mode.

16. The control apparatus for the medical image shooting apparatus according to claim 13, wherein the state detection sensor is a sensor configured to sense that the display apparatus is stored in a storage location of the display apparatus, and when the state detection sensor senses that the display apparatus is stored, the control unit performs the control for the switching from the second operation mode to the first operation mode.

17. The control apparatus for the medical image shooting apparatus according to claim 1, wherein the second touch operation is an operation of inputting a previously set character.

18. The control apparatus for the medical image shooting apparatus according to claim 1, wherein the medical image shooting apparatus is an ophthalmic shooting apparatus.

19. A medical image shooting system comprising:
a medical image shooting apparatus; and
a control apparatus configured to control the medical image shooting apparatus, wherein
the control apparatus configured to control the medical image shooting apparatus is the control apparatus for the the control apparatus comprising:
a control terminal configured to control a medical image shooting apparatus that shoots a subject; and
a display apparatus which is capable of communicating with the control terminal and which is portable, wherein
the display apparatus includes
a touch panel unit configured to perform display for a touch operation and accept a touch operation,
a signal generation unit configured to generate a drive instruction signal for instructing drive of a drive unit included in the medical image shooting apparatus based on the touch operation accepted by the touch panel unit, and
a signal transmission unit configured to transmit the drive instruction signal to the control terminal that controls the medical image shooting apparatus,
the control terminal includes
a signal reception unit configured to receive the drive instruction signal, and
a control unit configured to perform control to cause the drive unit of the medical image shooting apparatus to drive based on the drive instruction signal received by the signal reception unit,
the touch panel unit accepts a first touch operation and a second touch operation,
the first touch operation is a touch operation of enabling an acceptance of the second touch operation, and
the signal transmission unit transmits, based on the second touch operation accepted by the touch panel unit, a drive instruction signal for changing a relative position of the subject and the medical image shooting apparatus to the control terminal.
